(19) United States
(12) Patent Application Publication
(10) Pub. No.: US 2023/0355526 A1
(43) Pub. Date: Nov. 9, 2023

(54) LIPID COMPOSITIONS COMPRISING POLYNUCLEOTIDE ANTIGENS

(71) Applicant: IMMUNOVACCINE TECHNOLOGIES INC., Dartmouth (CA)

(72) Inventors: Marianne STANFORD, Upper Tantallon (CA); Frederic ORS, Quebec (CA); Olga HRYTSENKO, Halifax (CA); Rajkannan RAJAGOPALAN, Dartmouth (CA)

(21) Appl. No.: 18/028,617

(22) PCT Filed: Sep. 28, 2021
(86) PCT No.: PCT/IB2021/000650

§ 371 Date: Mar. 27, 2023

Related U.S. Application Data
(60) Provisional application No. 63/084,179, filed on Sep. 28, 2020.

(51) Int. Cl. A61K 9/127; A61K 31/7088; A61K 47/36

(52) U.S. Cl. CPC A61K 9/1272; A61K 31/7088; A61K 47/36

(57) ABSTRACT

The present disclosure provides liposomal compositions comprising lipids, in particular phospholipids and cholesterol, a negatively charged biomolecule, such as a polynucleotide, an ionizable aminoglycoside, such as chitosan, and an oil-based carrier. The compositions can be used for delivery of the biomolecule to targeted cells. The disclosure also provides the use of the composition for the treatment or prevention of cancer or infectious disease or ailment ameliorated by humoral and cellular immune response. The compositions can also be used for expressing polypeptides encoded by the nucleic acid components in the targeted cells.

### CROSS-REFERENCE TO RELATED APPLICATIONS

This application claims priority to U.S. Provisional Application No. 63/084,179, filed Sep. 28, 2020, the disclosure of which is herein incorporated by reference in its entirety.

### FIELD OF THE INVENTION

The present invention relates to lipids compositions for delivery of negatively charged molecules such as a polynucleotide. The present invention also relates to use of such compositions for delivering a negatively charged molecule (e.g., polynucleotide) to a subject.

### BACKGROUND

Effective delivery of nucleic acids into target cells remains a key problem preventing the clinical application of nucleic acid-based therapies. Such nucleic acids may be, for example, sequences encoding a gene product or instead short sequences of nucleotides that correspond to the sense or antisense sequence of specific genes or their products and hence have a direct effect on the expression of these genes and/or their products.

Nucleic acid delivery presents unique challenges due to their molecular size, electric charge, and significant susceptibility to enzymatic degradation. There continues to exist problems in delivering nucleic acids to the correct target site and to a sufficient number of target cells. A wide variety of delivery methods have been proposed, including microinjection, scrape loading, and receptor-mediated endocytosis. Lipid-based delivery systems, including those involving the use of liposomes, are frequently used to package therapeutic nucleic acids. However, the use of lipids alone may pose problems such as poor encapsulation efficacy and rapid clearance from circulation. There may also be problems in packaging enough nucleic acid molecules without increasing the size of the particle to the point where delivery to the target tissues is impaired.

Accordingly, there exists a need to improve lipid-based delivery systems for efficient delivery of nucleic acids to the correct target site.

### SUMMARY OF THE INVENTION

The present disclosure provides, among other things, lipid compositions suitable for the delivery of negatively charged molecules (e.g., polynucleotides). Methods of using such lipid compositions for delivering a negatively charged molecule to a target cell or a subject, or for treatment of a disorder or a disease are also provided. Further provided are methods of preparing the lipid compositions and kits comprising the lipid compositions.

In one aspect, provided herein is a composition, comprising:
a) one or more lipids,
b) a negatively charged molecule,
c) a carrier comprising a continuous phase of a hydrophobic substance, and
d) an ionizable aminoglycoside.

In some embodiments, the lipids comprise one or more of a phospholipid, cholesterol or a cholesterol derivative, or a combination thereof. In some embodiments, the phospholipid is one or more of phosphoglycerol, phosphoethanolamine, phosphoserine, phosphocholine, phosphoinositol, phosphatidylcholine or lecithin. In some embodiments, the phospholipid comprises dioleoyl phosphatidylcholine (DOPC), 1,2-dipalmitoyl-sn-glycero-3-phosphocholine (DPPC), dioleoyl phosphatidylethanolamine (DOPE), 1,2-dipalmitoyl-sn-glycero-3-succinate (DGS), or a combination thereof. In some embodiments, the one or more lipids comprise DOPC and cholesterol.

In some embodiments, the ionizable aminoglycoside is one or more of chitosan, cationic alginate, cationic gelatin, cationic dextran, DEAE-dextran hydrochloride, aminated cellulose, aminated sucrose, aminated trehalose, N-acetyl-D-glucosamine, D-(+)-glucosamine hydrochloride, trehalose-6,6-dibehenate (TDB) with Dimethyldioctadecylammonium bromide (DDA), heptakis(6-deoxy-6-amino)-β-cyclodextrin heptahydrochloride, and glycyrrhizic acid ammonium salt, or derivatives thereof.

In some embodiments, the ionizable aminoglycoside is chitosan. In some embodiments, the chitosan has a molecular weight of about 60 kDa to 150 kDa (e.g., about 60 kDa, about 70 kDa, about 80 kDa, about 90 kDa, about 100 kDa, about 110 kDa, about 120 kDa, about 130 kDa, about 140 kDa, or about 150 kDa). In some embodiments, the chitosan has a molecular weight of about 100 kDa to 120 kDa. In one embodiment, the chitosan has a molecular weight of about 100 kDa. In some embodiments, the chitosan has a degree of deacetylation (DD) of about 15-95%. In one embodiment, the chitosan has a degree of deacetylation (DD) of about 25%.

In some embodiments, the chitosan is added in a concentration of about 0.5 mg/mL to about 3 mg/mL (e.g., about 0.5 mg/mL, about 0.75 mg/mL, about 1 mg/mL, about 1.25 mg/mL, about 1.5 mg/mL, about 1.75 mg/mL, about 2 mg/mL, about 2.25 mg/mL, about 2.5 mg/mL, about 2.75 mg/mL or about 3 mg/mL). In some embodiments, the chitosan is added in a concentration of about 1 mg/mL to about 2 mg/mL.

In some embodiments, the composition described above further comprises an adjuvant.

In some embodiments, provided herein is a composition, comprising:
a) one or more positively charged lipids,
b) a negatively charged molecule,
c) a carrier comprising a continuous phase of a hydrophobic substance,
d) an ionizable aminoglycoside, and
e) optionally, an adjuvant.

In some embodiments, the one or more positively charged lipids comprise 1,2-dioleoyl-3-trimethylammonium-propane (DOTAP), 30-[N—(N′,N′-dimethylaminoethane)-carbamoyl]cholesterol (DC-cholesterol), 1,2-distearoyl-3-dimethylammonium-propane (DAP), N-(4-carboxybenzyl)-N,N-dimethyl-2,3-bis(oleoyloxy)propan-1-aminium (DOBAQ), N-palmitoyl homocysteine (PHC), DC-cholesterol, or a combination thereof.

In some embodiments, the negatively charged molecule is a polynucleotide. In some embodiments, the negatively charged molecule is a ribonucleic acid (RNA), or RNA derivative. In some embodiments, the negatively charged molecule is a deoxyribonucleic acid (DNA), or DNA derivative. In some embodiments, the polynucleotide comprises or encodes a messenger RNA (mRNA), an antisense RNA, an interfering RNA, a catalytic RNA, or a ribozyme. In some embodiments, the polynucleotide comprises an mRNA.

In some embodiments, the polynucleotide encodes a polypeptide. In some embodiments, the polypeptide is an antigen, an antibody or antibody fragment, an enzyme, a cytokine, a therapeutic protein, a chemokine, a regulatory protein, a structural protein, a chimeric protein, a nuclear protein, a transcription factor, a viral protein, a TLR protein, an interferon regulatory factor, an angiostatic or angiogenic protein, an apoptotic protein, an Fc gamma receptor, a hematopoietic protein, a tumor suppressor, a cytokine receptor, or a chemokine receptor. In some embodiments, the antigen is derived from a virus, bacterium or protozoan, a membrane surface-bound cancer antigen, a toxin, or an allergen.

In some embodiments, the concentration ratio of the lipids and the negatively charged molecule is between about 33000:1 to about 3300:1. In some embodiments, the concentration ratio of the lipids and the negatively charged molecule is between about 30800:1 to about 4400:1. In some embodiments, the concentration ratio of the lipids and the negatively charged molecule is between about 28600:1 to about 5500:1. In some embodiments, the concentration ratio of the lipids and the negatively charged molecule is between about 26400:1 to about 6600:1. In some embodiments, the concentration ratio of the lipids and the negatively charged molecule is between about 24200:1 to about 7700:1. In some embodiments, the concentration ratio of the lipids and the negatively charged molecule is between about 22000:1 to about 8800:1. In some embodiments, the concentration ratio of the lipids and the negatively charged molecule is between about 19800:1 to about 9900:1. In some embodiments, the concentration ratio of the lipids and the negatively charged molecule is between about 17600:1 to about 11000:1. As a non-limiting example, the concentration ratio of the lipids and the negatively charged molecule may be about 4400:1, 5500:1, 6600:1, 7700:1, 8800:1, 9900:1, 11000:1, 12100:1, 13200:1, 14300:1, 15400:1, 16500:1, 17600:1, 18700:1, 19800:1, 22000:1, 23100:1, 24200:1, 25300:1, 26400:1, 27500:1, 28600:1, 29700:1, 30800:1, 31900:1, or 33000:1. In one embodiment, the concentration ratio of the lipids and the negatively charged molecule is about 13200:1.

In some embodiments, the carrier comprises an oil or a water-in-oil emulsion.

In some embodiments of the composition,
a) the one or more lipids comprise DOPC and cholesterol,
b) the negatively charged molecule is a polynucleotide,
c) the carrier comprises an oil or a water-in-oil emulsion, and
d) the ionizable aminoglycoside is chitosan.

In some embodiments, the oil comprises a natural oil or a synthetic oil. In some embodiments, the oil comprises a vegetable oil, mineral oil, a nut oil, soybean oil, peanut oil, or combinations thereof. In some embodiments, the carrier comprises a mannide oleate in mineral oil solution. In some embodiments, the carrier comprises Montanide® ISA 51. In some embodiments, the carrier comprises MS80 oil (mixture of mineral oil and Span 80).

In some embodiments, the adjuvant is a polymer, a protein, a polysaccharide, or a combination thereof.

In some embodiments, the composition further comprises a buffer and/or surfactant.

In some embodiments, the composition is an injectable composition.

In another aspect, provided herein is a method for delivering a negatively charged molecule to a target cell, comprising administering the composition of any one of the embodiments above to said target cell. In some embodiments, the target cell is an antigen-presenting cell (APC).

In another aspect, provided herein is a method for delivering a negatively charged molecule to a subject, comprising administering the composition of any one of the embodiments above to said subject.

In another aspect, provided herein is a method for treating or preventing cancer, an infectious disease or an disease and/or disorder ameliorated by humoral and/or cellular immune response in a subject in need thereof, said method comprising administering to the subject an effective amount of the composition of composition of any one of the embodiments above. In some embodiments, the negatively charged molecule is a polynucleotide. In some embodiments, the polynucleotide comprises an mRNA. In some embodiments, the polynucleotide encodes a polypeptide. In some embodiments, the polypeptide is an antigen, an antibody or antibody fragment, an enzyme, a cytokine, a therapeutic protein, a chemokine, a regulatory protein, a structural protein, a chimeric protein, a nuclear protein, a transcription factor, a viral protein, a TLR protein, an interferon regulatory factor, an angiostatic or angiogenic protein, an apoptotic protein, an Fc gamma receptor, a hematopoietic protein, a tumor suppressor, a cytokine receptor, or a chemokine receptor. In some embodiments, the antigen is derived from a virus, bacterium or protozoan, a membrane surface-bound cancer antigen or a toxin. In some embodiments, the composition is administered via subcutaneous, intramuscular or intradermal injection.

In another aspect, provided herein is a method for preparing a composition of any of the embodiments described above.

In another aspect, provided herein is a method for preparing a composition comprising one or more lipids, a negatively charged molecule, a carrier comprising a continuous phase of a hydrophobic substance, and an ionizable aminoglycoside, comprising:
a) dissolving the one or more lipids in one or more organic solvents and optionally an aqueous solvent to create a lipid solution,
b) adding the negatively charged molecule to the lipid solution formed in step a) and mixing;
c) adding the ionizable aminoglycoside to the mixture formed in step b) and mixing;
d) optionally, adding additional amount of the organic solvent(s) or aqueous solvent to the mixture formed in step c) thereby the overall Wt/Wt or V/V percentage ratio of organic:aqueous solvent or aqueous:organic solvent in the mixture is between 20-50%;
e) drying the mixture formed in step c) or d) to generate a dried preparation; and
f) dissolving the dried preparation in the carrier comprising a continuous phase of a hydrophobic substance, thereby generating said composition.

In some embodiments of the method of preparing a composition described herein, in step a) the one or more organic solvents is present in an amount sufficient to prevent the one or more lipids from forming lipid vesicle particles in the lipid solution. In some embodiments, the organic solvent is tert-butanol, ethanol, methanol, chloroform, or a mixture thereof. In some embodiments, the organic solvent is tert-butanol, tert-butanol-ethanol mixture or tert-butanol-chloroform mixture. In some embodiments, the lipid solution comprises about 30% tert-butanol.

In some embodiments of the method of preparing a composition described herein, the aqueous solvent is water or a buffer solution.

In some embodiments of the method of preparing a composition described herein, drying is performed by freeze-drying, spray freeze-drying, spray drying, or rotary evaporation.

In some embodiments of the method of preparing a composition described herein, the lipids comprise one or more of a phospholipid, cholesterol or a cholesterol derivative, or a combination thereof. In some embodiments, the phospholipid is one or more of phosphoglycerol, phosphoethanolamine, phosphoserine, phosphocholine, phosphoinositol, phosphatidylcholine or lecithin. In some embodiments, the phospholipid comprises dioleoyl phosphatidylcholine (DOPC), 1,2-dipalmitoyl-sn-glycero-3-phosphocholine (DPPC), dioleoyl phosphatidylethanolamine (DOPE), 1,2-dipalmitoyl-sn-glycero-3-succinate (DGS), or a combination thereof. In some embodiments, the one or more lipids comprise DOPC and cholesterol.

In some embodiments of the method of preparing a composition described herein, the ionizable aminoglycoside is one or more of chitosan, cationic alginate, cationic gelatin, cationic dextran, DEAE-dextran hydrochloride, aminated cellulose, aminated sucrose, aminated trehalose, N-acetyl-D-glucosamine, D-(+)-glucosamine hydrochloride, trehalose-6,6-dibehenate (TDB) with Dimethyldioctadecylammonium (DDA), heptakis(6-deoxy-6-amino)-β-cyclodextrin heptahydrochloride, and glycyrrhizic acid ammonium salt, or derivatives thereof.

In some embodiments of the method of preparing a composition described herein, the ionizable aminoglycoside is chitosan. In some embodiments, the chitosan has a molecular weight of about 60 kDa to 150 kDa (e.g., about 60 kDa, about 70 kDa, about 80 kDa, about 90 kDa, about 100 kDa, about 110 kDa, about 120 kDa, about 130 kDa, about 140 kDa, or about 150 kDa). In some embodiments, the chitosan has a molecular weight of about 100 kDa to 120 kDa. In one embodiment, the chitosan has a molecular weight of about 100 kDa. In some embodiments, the chitosan has a degree of deacetylation (DD) of about 15-95%. In one embodiment, the chitosan has a degree of deacetylation (DD) of about 25%.

In some embodiments of the method of preparing a composition described herein, the chitosan is added in a concentration of about 0.5 mg/mL to about 3 mg/mL (e.g., about 0.5 mg/mL, about 0.75 mg/mL, about 1 mg/mL, about 1.25 mg/mL, about 1.5 mg/mL, about 1.75 mg/mL, about 2 mg/mL, about 2.25 mg/mL, about 2.5 mg/mL, about 2.75 mg/mL or about 3 mg/mL). In some embodiments, the chitosan is added in a concentration of about 1 mg/mL to about 2 mg/mL.

In some embodiments of the method of preparing a composition described herein, the negatively charged molecule is a polynucleotide. In some embodiments, the negatively charged molecule is a ribonucleic acid (RNA), or RNA derivative. In some embodiments, the negatively charged molecule is a deoxyribonucleic acid (DNA), or DNA derivative. In some embodiments, the polynucleotide comprises or encodes a messenger RNA (mRNA), an antisense RNA, an interfering RNA, a catalytic RNA, or a ribozyme. In some embodiments, the polynucleotide comprises an mRNA.

In some embodiments of the method of preparing a composition described herein, the polynucleotide encodes a polypeptide. In some embodiments, the polypeptide is an antigen, an antibody or antibody fragment, an enzyme, a cytokine, a therapeutic protein, a chemokine, a regulatory protein, a structural protein, a chimeric protein, a nuclear protein, a transcription factor, a viral protein, a TLR protein, an interferon regulatory factor, an angiostatic or angiogenic protein, an apoptotic protein, an Fc gamma receptor, a hematopoietic protein, a tumor suppressor, a cytokine receptor, or a chemokine receptor. In some embodiments, the antigen is derived from a virus, bacterium or protozoan, a membrane surface-bound cancer antigen, a toxin, or an allergen.

In some embodiments of the method of preparing a composition described herein, the concentration ratio of the lipids and the negatively charged molecule is between about 33000:1 to about 3300:1. In some embodiments, the concentration ratio of the lipids and the negatively charged molecule is between about 30800:1 to about 4400:1. In some embodiments, the concentration ratio of the lipids and the negatively charged molecule is between about 28600:1 to about 5500:1. In some embodiments, the concentration ratio of the lipids and the negatively charged molecule is between about 26400:1 to about 6600:1. In some embodiments, the concentration ratio of the lipids and the negatively charged molecule is between about 24200:1 to about 7700:1. In some embodiments, the concentration ratio of the lipids and the negatively charged molecule is between about 22000:1 to about 8800:1. In some embodiments, the concentration ratio of the lipids and the negatively charged molecule is between about 19800:1 to about 9900:1. In some embodiments, the concentration ratio of the lipids and the negatively charged molecule is between about 17600:1 to about 11000:1. As a non-limiting example, the concentration ratio of the lipids and the negatively charged molecule may be about 4400:1, 5500:1, 6600:1, 7700:1, 8800:1, 9900:1, 11000:1, 12100:1, 13200:1, 14300:1, 15400:1, 16500:1, 17600:1, 18700:1, 19800:1, 22000:1, 23100:1, 24200:1, 25300:1, 26400:1, 27500:1, 28600:1, 29700:1, 30800:1, 31900:1, or 33000:1. In one embodiment, the concentration ratio of the lipids and the negatively charged molecule is about 13200:1.

In some embodiments of the method of preparing a composition described herein, the carrier comprises an oil or a water-in-oil emulsion.

In some embodiments of the method of preparing a composition described herein,
a) the one or more lipids comprise DOPC and cholesterol,
b) the negatively charged molecule is a polynucleotide,
c) the carrier comprises an oil or a water-in-oil emulsion, and
d) the ionizable aminoglycoside is chitosan.

In some embodiments, the oil comprises a natural oil or a synthetic oil. In some embodiments, the oil comprises a vegetable oil, mineral oil, a nut oil, soybean oil, peanut oil, or combinations thereof. In some embodiments, the carrier comprises a mannide oleate in mineral oil solution. In some embodiments, the carrier comprises Montanide® ISA 51. In some embodiments, the carrier comprises MS80 oil (mixture of mineral oil and Span 80).

In some embodiments of the method of preparing a composition described herein, the composition further comprises an adjuvant. In some embodiments, the adjuvant is a polymer, a protein, a polysaccharide, or a combination thereof.

In some embodiments of the methods of preparing a composition described herein, the composition further comprises a buffer and/or surfactant.

In some embodiments of the methods of preparing a composition described herein, the composition is an injectable composition.

In another aspect, provided herein is a kit comprising a composition of any one of the embodiments above, and instructions for using said composition to deliver a negatively charged molecule to a subject.

In various embodiments, the subject is a mammal. In some embodiments, the subject is a human.

These and other aspects of the present invention will be apparent to those of ordinary skill in the art in the following description, claims and drawings.

### BRIEF DESCRIPTION OF THE DRAWINGS

FIG. 1 depicts a schematic of the nucleic acid delivery using an exemplary lipid vesicle particle of the present disclosure.

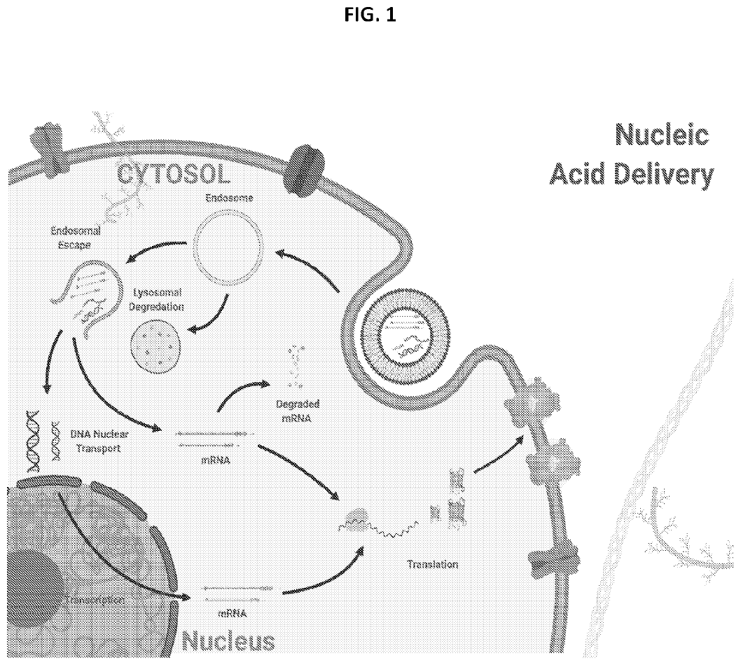

FIG. 2 shows green fluorescent protein (GFP) expression levels at the site of injection after administration of eGFP mRNA loaded lipid vesicle particles containing different polymers and transfection agents. Sample 1: formulation method A (standard) without addition of different polymers and transfection agents.

FIG. 3 shows GFP expression levels at the site of injection after administration of eGFP mRNA loaded lipid vesicle particles containing 1,2-dioleoyl-3-trimethylammonium-propane (DOTAP). Sample 1: formulation method A (standard) without addition of 1,2-dioleoyl-3-trimethylammonium-propane (DOTAP).

FIG. 4 shows GFP expression levels at the site of injection after administration of eGFP mRNA loaded lipid vesicle particles containing DC-Cholesterol. Sample 1: formulation method A (standard) without addition of DC-Cholesterol.

FIG. 5 shows GFP expression levels at the site of injection after administration of eGFP mRNA loaded lipid vesicle particles containing DOTAP. Sample 1: formulation method A (standard) without addition of DOTAP.

FIG. 6 shows GFP expression levels at the site of injection after administration of eGFP mRNA loaded lipid vesicle particles containing chitosan. Sample 1: formulation method A (standard) without addition of chitosan.

FIG. 7A-7B show antigen-specific interferon-gamma (IFN-γ) response in the spleens (FIG. 7A) and lymph nodes (FIG. 7B) collected from mice receiving the indicated lipid formulations. The IFN-γ response was determined using an enzyme-linked immunospot (ELISpot) assay.

FIG. 8A-8B show immune profiles of site of injections (SOIs) collected from mice receiving the indicated lipid formulations.

FIG. 9 shows the study design of an in vitro stability study of lipid-nucleic acid formulations.

FIG. 10A-10B show mRNA (FIG. 10A) and DNA (FIG. 10B) quantities in the investigated lipid-nucleic acid formulations as measured by UV spectroscopy.

FIG. 11A-11B show gel electrophoresis analyses of mRNA (FIG. 11A) and DNA (FIG. 11B) in the investigated lipid-nucleic acid formulations.

FIGS. 12A-12B show quantification of RNA or DNA expression efficiency (FIG. 12A) evaluated in transient transfection experiments as well as representative fluorescence microscopy images (FIG. 12B).

FIG. 13 shows the study design of an in vivo stability study of lipid-mRNA formulations.

FIGS. 14A-14B show that mRNA formulated in the investigated lipid-mRNA formulation is stable for up to 14 days in vivo. FIG. 14A shows evaluation of E7 mRNA integrity by detection of E7 transcripts using RT-PCR and gel electrophoresis. Complete sequence of the E7 transcript was amplified via RT-PCR from total RNA extracted from SOIs.

FIG. 14B shows quantification of GFP expression in cells transfected with total RNA extracted from SOIs.

### DETAILED DESCRIPTION

The present disclosure provides, among other things, lipid vesicle particles that facilitate delivery of molecules (e.g., polynucleotides) into a biological system, for example into cells such as mammalian cells.

### Definitions

As used herein, the term “lipid vesicle particle” may be used interchangeably with “lipid vesicle”. A lipid vesicle particle refers to a complex or structure having an internal environment separated from the external environment by a continuous layer of enveloping lipids. In the context of the present disclosure, the expression “layer of enveloping lipids” can mean a single layer lipid membrane (e.g., as found on a micelle or reverse micelle), a bilayer lipid membrane (e.g., as found on a liposome) or any multilayer membrane formed from single and/or bilayer lipid membranes. The layer of enveloping lipids is typically a single layer, bilayer or multilayer throughout its circumference, but it is contemplated that other conformations may be possible such that the layer has different configurations over its circumference. The lipid vesicle particle may contain, within its internal environment, other vesicle structures (i.e., it may be multivesicular).

The term “lipid vesicle particle” encompasses many different types of structures, including without limitation micelles, reverse micelles, unilamellar liposomes, multilamellar liposomes and multivesicular liposomes. The lipid vesicle particles may take on various different shapes, and the shape may change at any given time (e.g., upon sizing, mixing with the second therapeutic agent, and/or drying). Typically, lipid vesicle particles are spherical or substantially spherical structures. By “substantially spherical” it is meant that the lipid vesicle particles are close to spherical, but may not be a perfect sphere. Other shapes of the lipid vesicle particles include, without limitation, oval, oblong, square, rectangular, triangular, cuboid, crescent, diamond, cylinder or hemisphere shapes. Any regular or irregular shape may be formed. Further, a single lipid vesicle particle may comprise different shapes if it is multivesicular. For example, the outer vesicle shape may be oblong or rectangular while an inner vesicle may be spherical.

The lipid vesicle particles may be formed from single layer lipid membranes, bilayer lipid membranes and/or multilayer lipid membranes. The lipid membranes are predominantly comprised of and formed by lipids, but may also comprise additional components. For example, and without limitation, the lipid membrane may include stabilizing molecules to aid in maintaining the size and/or shape of the lipid vesicle particle. Any stabilizing molecule known in the art may be used so long as it does not negatively affect the ability of the lipid vesicle particles to be used in the disclosed methods.

Depending on the definition ascribed to lipid nanoparticles, the lipid vesicle particles of the present disclosure may be synonymous with lipid nanoparticles. However, there are contrasting views in the art on the meaning of the term “lipid nanoparticle”. One view is that a lipid nanoparticle refers to any nano-sized particle (i.e., having a diameter of between 1 nanometer and 1000 nanometers) formed by a lipid membrane. Another view is that the size threshold for a nanoparticle material is limited to between 1 nanometer and 100 nanometers. This latter definition excludes lipid vesicle sizes that are encompassed by the present disclosure (e.g., lipid vesicle particles >100 nm in size), and to this extent is inconsistent with the term “lipid vesicle particles” as used in the present disclosure.

The term “lipid” has its common meaning in the art in that it is any organic substance or compound that is soluble in nonpolar solvents, but generally insoluble in polar solvents (e.g., water). Lipids are a diverse group of compounds including, without limitation, fats, waxes, sterols, fat-soluble vitamins, monoglycerides, diglycerides, triglycerides and phospholipids. For the lipid vesicle particles herein, any lipid may be used so long as it is a membrane-forming lipid. The lipid vesicle particles may comprise a single type of lipid or two or more different types of lipids.

The term “negatively charged molecule” as used herein, includes molecules such as naturally occurring and chemically modified nucleic acid molecules (e.g., RNA, DNA, polynucleotides, oligonucleotides, mixed polymers, peptide nucleic acid, and the like), peptides (e.g., polyaminoacids, polypeptides, proteins and the like), nucleotides, pharmaceutical and biological compositions, that have negatively charged groups.

As used herein the term “polynucleotide” encompasses a chain of nucleotides of any length (e.g., 9, 12, 18, 24, 30, 60, 150, 300, 600, 1500, 2000, 2500, 3000, 4000, 5000, 6000, 7000, 8000 or more nucleotides) or number of strands (e.g., single-stranded or double-stranded). Polynucleotides may be DNA (e.g., genomic DNA or cDNA) or RNA (e.g., mRNA, siRNA, miRNA, shRNA, self-amplifying RNA) or combinations thereof. They may be naturally occurring or synthetic (e.g., chemically synthesized). It is contemplated that the polynucleotide may contain modifications of one or more nitrogenous bases, pentose sugars or phosphate groups in the nucleotide chain. Such modifications are well-known in the art and may be for the purpose of e.g., improving stability of the polynucleotide.

As used herein, the term “polypeptide” or “protein” means any chain of amino acids, regardless of length (e.g., 4, 6, 8, 10, 20, 50, 100, 200, 500 or more amino acids) or post-translational modification (e.g., glycosylation or phosphorylation). Both terms are used interchangeably.

The term “ionizable aminoglycoside” refers to any polyamino sugar having the ability to bind with a negatively charged molecule (e.g., polynucleotide such as RNA) through electrostatic interactions. The polyamino sugar may be naturally occurring, semi-synthetic, or fully synthetic. Through interaction with the negatively charged molecule such as a polynucleotide (e.g., RNA), the ionizable aminoglycoside may protect the polynucleotide (e.g., RNA) from nuclease attack.

The term “adjuvant” refers to a compound or mixture that enhances the immune response to an antigen.

As used herein, the term “antigen” refers to any substance or molecule that can bind specifically to components of the immune system. In some embodiments, suitable antigens are those that are capable of inducing or generating an immune response in a subject. An antigen that is capable of inducing an immune response is said to be immunogenic, and may also be called an immunogen. Thus, as used herein, the term “antigen” includes immunogens and the terms may be used interchangeably unless specifically stated otherwise.

A “toxin”, as used herein, refers to any substance produced by living cells or organisms (e.g., plants, animals, microorganisms, etc.) that is capable of causing a disease or ailment, or an infectious substance, or a recombinant or synthesized molecule capable of adverse effect. Toxins may be for example small molecules (e.g., cocaine), peptides, or proteins.

An “allergen”, as used herein, refers to any substance that can cause an allergy.

An “antibody” is a protein comprising one or more polypeptides substantially or partially encoded by immunoglobulin genes or fragments of immunoglobulin genes. The recognized immunoglobulin genes include the x, λ, α, γ, δ, ε and μ constant region genes, as well as myriad immunoglobulin variable region genes. Light chains are classified as either κ or λ. Heavy chains are classified as γ, μ, α, δ, or ε, which in turn define the immunoglobulin classes, IgG, IgM, IgA, IgD and IgE, respectively. A typical immunoglobulin (antibody) structural unit comprises a protein containing four polypeptides. Each antibody structural unit is composed of two identical pairs of polypeptide chains, each having one “light” and one “heavy” chain. The N-terminus of each chain defines a variable region primarily responsible for antigen recognition. Antibody structural units (e.g., of the IgA and IgM classes) may also assemble into oligomeric forms with each other and additional polypeptide chains, for example as IgM pentamers in association with the J-chain polypeptide.

As used herein, the terms “cancer”, “cancer cells”, “tumor” and “tumor cells”, (used interchangeably) refer to cells that exhibit abnormal growth, characterized by a significant loss of control of cell proliferation or cells that have been immortalized. The term “cancer” or “tumor” includes metastatic as well as non-metastatic cancer or tumors. A cancer may be diagnosed using criteria generally accepted in the art, including the presence of a malignant tumor.

“Treating” or “treatment of”, or “preventing” or “prevention of”, as referred to herein refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, conferring protective immunity against a disease-causing agent and amelioration or palliation of the disease state. “Treating” or “preventing” can also mean prolonging survival of a patient beyond that expected in the absence of treatment and can also mean inhibiting the progression of disease temporarily, although more preferably, it involves preventing the occurrence of disease such as by preventing infection in a subject.

The term “effective amount” as used herein means an amount effective, at dosages and for periods of time necessary, to achieve the desired result.

As used herein, to “induce” an immune response is to elicit and/or potentiate an immune response. Inducing an immune response encompasses instances where the immune response is enhanced, elevated, improved or strengthened to the benefit of the host relative to the prior immune response status, for example, before the administration of a composition of the disclosure.

As used herein, the term “antibody response” refers to an increase in the amount of antigen-specific antibodies in the body of a subject in response to introduction of the antigen into the body of the subject.

“Humoral immune response” as referred to herein relates to antibody production and the accessory processes that accompany it, such as for example T-helper 2 (Th2) cell activation and cytokine production, isotype switching, affinity maturation and memory cell activation. It also refers to the effector functions of an antibody, such as for example toxin neutralization, classical complement activation, and promotion of phagocytosis and pathogen elimination. The humoral immune response is aided by CD4+Th2 cells and therefore the activation or generation of this cell type is also indicative of a humoral immune response as referred to herein.

A “humoral immune response” as referred to herein may also encompass the generation and/or activation of T-helper 17 (Th17) cells. Th17 cells are a subset of helper-effector T-lymphocytes characterized by the secretion of host defense cytokines such as IL-17, IL-17F, IL-21, and IL-22. Th17 cells are considered developmentally distinct from Th1 and Th2 cells, and have been postulated to facilitate the humoral immune response, such as for example, providing an important function in anti-microbial immunity and protecting against infections. Their production of IL-22 is thought to stimulate epithelial cells to produce anti-microbial proteins and production of IL-17 may be involved in the recruitment, activation and migration of neutrophils to protect against host infection by various bacterial and fungal species.

As used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

As used throughout herein, the term “about” means reasonably close. For example, “about” can mean within an acceptable standard deviation and/or an acceptable error range for the particular value as determined by one of ordinary skill in the art, which will depend on how the particular value is measured. Further, when whole numbers are represented, about can refer to decimal values on either side of the whole number. When used in the context of a range, the term “about” encompasses all of the exemplary values between the one particular value at one end of the range and the other particular value at the other end of the range, as well as reasonably close values beyond each end.

### Compositions of the Invention

In one aspect, provided herein is a composition comprising one or more lipids, a negatively charged molecule, a carrier comprising a continuous phase of a hydrophobic substance, and an ionizable aminoglycoside.

In another aspect, provided herein is a composition comprising one or more positively charged lipids (or cationic lipids), a negatively charged molecule, a carrier comprising a continuous phase of a hydrophobic substance, and optionally an adjuvant.

### Negatively Charged Molecules

Negatively charged molecules that can be delivered using the composition of the present disclosure include, but are not limited to, naturally occurring and chemically modified nucleic acid molecules (e.g., RNA, DNA, polynucleotides, oligonucleotides, mixed polymers, peptide nucleic acid, and the like), peptides (e.g., polyaminoacids, polypeptides, proteins and the like), nucleotides, pharmaceutical and biological compositions, that have negatively charged groups. While not wishing to be bound by theory, the negatively charged groups may ion-pair with the positively charged groups of any positively charged molecules (e.g., lipids, adjuvant) in the lipid vesicle particles of the disclosure.

In some embodiments, the negatively charged molecule in the composition of the present disclosure is a polynucleotide.

The use of polynucleotides as described herein refers specifically to polynucleotides that contain sequences that correspond largely to the sense or antisense sequence of specific genes or their products, and hence have a direct effect on the expression of these genes and/or their products. For example, the use of polynucleotides that contain gene coding sequences affects the transcription and/or translation of the genes of interest in cells that uptake such polynucleotides. Similarly, the use of RNA interference polynucleotides affects the expression of specific genes of interest by directly affecting the levels of mRNA in cells that uptake such nucleotides. This differs significantly from other polynucleotide-based molecules such as CpG and polyIC adjuvants, which do not act through the presence of gene specific sequences. Furthermore, polynucleotide-based adjuvants are believed to modulate an immune response in a non-specific manner, and their actions start at the site of vaccination where they interact with extracellular receptors to enhance the activity of immune cells in a non-specific manner. In some cases, polynucleotide-based adjuvants are internalized whereby they exert their effects by interacting with intracellular receptors, similarly leading to the activation of downstream pathways, and resulting collectively in the enhancement of immune cell activity to aid in the generation of an immune response. Such adjuvants do not directly affect the expression of specific genes that are being targeted by polynucleotide constructs as contemplated herein. Such adjuvants do not directly interact with the expression products of targeted genes, nor do they contain sequences that correspond to the sense or antisense sequence of targeted genes.

In some embodiments, the composition is useful for enhancing the expression of a polypeptide-encoding polynucleotide in vivo. For example, the polynucleotide encode a polypeptide that is deficient in the subject. In other embodiments, the polynucleotide may not encode a polypeptide, but may instead be e.g., a polynucleotide comprising or encoding an antisense RNA or other molecule that is not a polypeptide.

In some embodiments, the compositions comprise a polynucleotide of interest, optionally operably linked to regulatory sequences suitable for directing protein expression from the polynucleotide (e.g., a promoter), lipids, and a carrier comprising a continuous phase of a hydrophobic substance.

The compositions of the disclosure are useful for delivering polynucleotides of all kinds to a subject in vivo. In some embodiments, the polynucleotide is not expressed as a protein in the subject, but rather encodes e.g., an antisense RNA, an interfering RNA, a catalytic RNA, or a ribozyme. In some embodiments, the polynucleotide encodes a polypeptide to be expressed in vivo in a subject. In one embodiment, the polynucleotide is a messenger RNA (mRNA). The disclosure is not limited to the expression of any particular type of polypeptide. The polypeptide may be, merely by way of illustrative examples, an antigen, an antibody or antibody fragment, an enzyme, a cytokine, a therapeutic protein, a chemokine, a regulatory protein, a structural protein, a chimeric protein, a nuclear protein, a transcription factor, a viral protein, a TLR protein, an interferon regulatory factor, an angiostatic or angiogenic protein, an apoptotic protein, an Fc gamma receptor, a hematopoietic protein, a tumor suppressor, a cytokine receptor, or a chemokine receptor.

In some embodiments, the polynucleotides delivered with the compositions of the present disclosure may encode an antigen such as, but not limited to: those derived from Cholera toxoid, tetanus toxoid, diphtheria toxoid, hepatitis B surface antigen, hemagglutinin, neuraminidase, influenza M protein, PfHRP2, pLDH, aldolase, MSP1, MSP2, AMA1, Der-p-1, Der-f-1, Adipophilin, AFP, AIM-2, ART-4, BAGE, alpha-fetoprotein, BCL-2, Bcr-Abl, BING-4, CEA, CPSF, CT, cyclin D1Ep-CAM, EphA2, EphA3, ELF-2, FGF-5, G250, Gonadotropin Releasing Hormone, HER-2, intestinal carboxyl esterase (iCE), IL13Ralpha2, MAGE-1, MAGE-2, MAGE-3, MART-1, MART-2, M-CSF, MDM-2, MMP-2, MUC-1, NY-EOS-1, MUM-1, MUM-2, MUM-3, p53, PBF, PRAME, PSA, PSMA, RAGE-1, RNF43, RU1, RU2AS, SART-1, SART-2, SART-3, SAGE-1, SCRN 1, SOX2, SOX10, STEAP1, survivin (BIRC5), Telomerase, TGFbetaR11, TRAG-3, TRP-1, TRP-2, TERT, or WT1; those derived from a virus, such as Cowpoxvirus, Vaccinia virus, Pseudocowpox virus, Human herpesvirus 1, Human herpesvirus 2, Cytomegalovirus, Human adenovirus A-F, Polyomavirus, Human papillomavirus, Parvovirus, Hepatitis A virus, Hepatitis B virus, Hepatitis C virus, Human immunodeficiency virus, Orthoreovirus, Rotavirus, Ebolavirus, parainfluenza virus, influenza virus (e.g., H5N1 influenza virus, influenza A virus, influenza B virus, influenza C virus). Measles virus, Mumps virus, Rubella virus, Pneumovirus, Human respiratory syncytial virus, Rabies virus, California encephalitis virus, Japanese encephalitis virus, Hantaan virus, Lymphocytic choriomeningitis virus, Coronavirus (e.g., SARS-CoV-2), Enterovirus, Rhinovirus, Poliovirus, Norovirus, Flavivirus, Dengue virus, West Nile virus, Yellow fever virus and varicella; those derived from a bacterium, such as Anthrax (Bacillus anthracis), Brucella, Bordetella pertussis, Candida, Chlamydia pneumoniae, Chiamydia psittaci, Cholera, Clostridium botulinum, Coccidioides immitis, Cryptococcus, Diphtheria, Escherichia coli O157: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica; or those derived from a protozoa, e.g., of the genus Plasmodium (Plasmodium falciparum, Plasmodium malariae, Plasmodium vivax, Plasmodium ovale or Plasmodium knowlesi).

The antigen may be an allergen derived from, without limitation, cells, cell extracts, proteins, polypeptides, peptides, peptide mimics of polysaccharides and other molecules, such as small molecules, lipids, glycolipids, and carbohydrates of plants, animals, fungi, insects, food, drugs, dust, and mites. Allergens include but are not limited to environmental aeroallergens; plant pollens (e.g., ragweed/hayfever); weed pollen allergens; grass pollen allergens; Johnson grass; tree pollen allergens; ryegrass; arachnid allergens (e.g., house dust mite allergens); storage mite allergens; Japanese cedar pollen/hay fever; mold/fungal spore allergens; animal allergens (e.g., dog, guinea pig, hamster, gerbil, rat, mouse, etc., allergens); food allergens (e.g., crustaceans; nuts; citrus fruits; flour; coffee); insect allergens (e.g., fleas, cockroach); venoms: (Hymenoptera, yellow jacket, honey bee, wasp, hornet, fire ant); bacterial allergens (e.g., streptococcal antigens; parasite allergens such as Ascaris antigen); viral antigens; drug allergens; hormones (e.g., insulin); enzymes (e.g., streptokinase); and drugs or chemicals capable of acting as incomplete antigens or haptens (e.g., the acid anhydrides and the isocyanates). Where a hapten is used in a composition of the disclosure, it may be attached to a carrier to form a hapten-carrier adduct. The hapten-carrier adduct is capable of initiating a humoral immune response, whereas the hapten itself would not elicit antibody production. Non-limiting examples of haptens are aniline, urushiol (a toxin in poison ivy), hydralazine, fluorescein, biotin, digoxigenin and dinitrophenol.

In another embodiment, the antigen may be an antigen associated with a disease where it is desirable to sequester the antigen in circulation, such as for example an amyloid protein (e.g., Alzheimer's disease).

In some embodiments, the polynucleotide may encode a T-helper epitope. T helper epitope is a sequence of amino acids (natural or non-natural amino acids) that have T helper activity. T helper epitopes are recognized by T helper lymphocytes, which play an important role in establishing and maximizing the capabilities of the immune system, and are involved in activating and directing other immune cells, such as for example B cell antibody class switching.

A T-helper epitope can consist of a continuous or discontinuous epitope. Hence not every amino acid of a T-helper is necessarily part of the epitope. Accordingly, T-helper epitopes, including analogs and segments of T-helper epitopes, are capable of enhancing or stimulating an immune response. Immunodominant T-helper epitopes are broadly reactive in animal and human populations with widely divergent MHC types (Celis et al. (1988) J. Immunol. 140:1808-1815; Demotz et al. (1989) J. Immunol. 142:394-402; Chong et al. (1992) Infect. lmmun. 60:4640-4647). The T-helper domain of the subject peptides has from about 10 to about 50 amino acids and preferably from about 10 to about 30 amino acids. When multiple T-helper epitopes are present, then each T-helper epitope acts independently.

In some embodiments, the T-helper epitope may be encoded in a polynucleotide as part of an antigen described herein. In particular, if the antigen is of sufficient size, it may contain an epitope that functions as a T-helper epitope. In other embodiments, the T-helper epitope is encoded in a polynucleotide as a separate molecule from the antigen. In some embodiments, the T helper epitope is encoded in a polynucleotide with at least one antigen (i.e., a peptide), or a mixture of antigens, to make a fusion peptide.

In another embodiment, T-helper epitope analogs may include substitutions, deletions and insertions of from one to about 10 amino acid residues in the T-helper epitope. T-helper segments are contiguous portions of a T-helper epitope that are sufficient to enhance or stimulate an immune response. An example of T-helper segments is a series of overlapping peptides that are derived from a single longer peptide.

RNA interference (RNAi) is a sequence specific post-transcriptional gene silencing mechanism, which is triggered by double-stranded RNA such as small (or short) interference RNA (siRNA) and single stranded intracellular RNA such as microRNA (miRNA), both of which can cause degradation of mRNAs homologous in sequence to siRNA or miRNA (Fire et al, 1998, Nature, 391:806-811; Montgomery et al, 1998, PNAS, 95:15502-15507; Elbashir et al, 2001, Nature, 411:494-498). RNAi is a conserved pathway common to plants and mammals that suppress expression of genes with complementary sequences (Hannon and Rossi, 2004, Nature, 431:371-378; Meister and Tuschl, 2004, Nature, 431, 343-349). RNAi was first observed in lower organisms, such as plants or nematodes. In these systems, long dsRNAs serve as effective triggers of RNAi. Long dsRNAs are not the actual triggers but are degraded by the endoribonuclease Dicer into small effector molecules called siRNAs. In mammals, Dicer processing occurs as a complex with the RNA-binding protein TRBP. The nascent siRNA associates with Dicer, TRBP, and Ago2 to form the RNA-Induced Silencing Complex (RISC) that mediates gene silencing (Chendrimada et al, 2005, Nature, 436:740-744). Once in RISC, one strand of the siRNA (the passenger strand) is degraded or discarded while the other strand (the guide strand) remains to direct sequence specificity of the silencing complex. The Ago2 component of RISC is a ribonuclease that cleaves a target RNA under direction of the guide strand.

Although long dsRNAs (several hundred bp) are commonly employed to trigger RNAi in C. elegans or D. melanogaster, these molecules will activate the innate immune system and trigger interferon (IFN) responses in higher organisms. RNAi can be performed in mammalian cells using short RNAs, which generally do not induce IFN responses. Many researchers today employ synthetic 21-mer RNA duplexes as their RNAi reagents, which mimic the natural siRNAs that result from Dicer processing of long substrate RNAs. An alternative approach is to use synthetic RNA duplexes that are greater than 21-mer in length, which are substrates for Dicer (Tuschl, T. 2002, Nature Biotechnology, 20:446).

Recently developed Dicer-substrate RNAs (DsiRNAs) are chemically synthesized RNA duplexes that have increased potency in RNA interference (Kim et al, 2005, Nat Biotechnol, 23:222-226). DsiRNAs are processed by Dicer into 21-mer siRNAs and designed so that cleavage results in a single, desired product. This is achieved through use of a novel asymmetric design where the RNA duplex has a single 2-base 3′-overhang on the AS strand and is blunt on the other end; the blunt end is modified with DNA bases. This design provides Dicer with a single favorable PAZ binding site that helps direct the cleavage reaction. Functional polarity is introduced by this processing event, which favors AS strand loading into RISC, and the increased potency of these reagents is thought to relate to linkage between Dicer processing and RISC loading (Rose et al, 2005, Nucleic Acids Res, 33:4140-4156). The Dicer-substrate approach can result in reagents having as much as 10-fold higher potency than traditional 21-mer siRNAs at the same site. miRNA, first described in 1993 (Lee et al, 1993, Cell 75:843-854), are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression. Although miRNA is generated within the cell and is highly conserved, it rarely has perfect complementarity with mRNA sequences. However, miRNA can affect protein translation and mRNA decay by binding to its imperfectly matched target sites on 3′ UTR region of mRNA, which also requires Ago protein (not necessarily Ago2 as in seen in siRNA). Comparing and contrasting siRNA with miRNA shows that if siRNA hits an imperfect complementary target on 3 UTR, it behaves similar to microRNA, and if a miRNA hits a perfectly matched target on a mRNA, it can behave like an siRNA. Hence, although structurally different, both siRNA and miRNA might possess similar biological functions in the cells of host animal, with some differences in the mechanism of action.

Thus, RNAi molecules, siRNA and/or miRNA, provide a powerful tool for inhibiting endogenous gene expression and thereby could provide a means to effectively modulate biological responses. Studies have shown that RNAi can be induced in antigen presenting dendritic cells (DC) to polarize immune responses. By transfecting DC with synthetic siRNA specific for cytokine IL-12 p35 sub-unit, it was possible to inhibit bioactive IL-12 which subsequently led to Th2 polarization (Hill et al, J Immunology, 2003, 171:691). Similarly, modification of professional antigen presenting cells with siRNA in vivo has been used to enhance cancer vaccine potency (Kim et al, Cancer Research, 2005, 65:309-316). In this study, co-administration of DNA vaccine encoding human papilloma virus type 16 E7 with siRNA targeting key pro-apoptotic proteins Bac and Bax was shown to prolong the life span of antigen expressing DCs in the lymph nodes, enhancing antigen-specific CD8 T cell responses that had potent anti-tumor effects against an E7-expressing tumor model in vaccinated mice. Thus, there is a good prospect for the use of siRNA for silencing specific undesirable responses during effective vaccination against infectious/autoimmune diseases, cancer and during transplantation. Efficient delivery of siRNA to the intracellular compartment of cells of interest is critical for the success of such strategies, requiring the use of enhanced delivery formulations.

siRNA may be a naturally occurring or synthetic double stranded nucleotide (RNA) chain of varying length. siRNA can be duplexes, usually but not always limited to, 20 to 25-nt long that have 19 base pair central double stranded domain with terminal 2-base 3′ overhangs. siRNA can be further modified chemically to enhance its in vivo efficacy, induce nuclease-resistance to prevent degradation and enhance stability. In this regard, the anti-sense strand may have either a free 5′-OH or 5′-phosphate terminus, the latter results in natural Dicer processing and represents the active form of the molecule. siRNA may have phosphorothioate or boranohosphate modification of the internucleoside linkage to improve nuclease stability and prolong life of the duplex when exposed to serum or other nuclease sources. siRNA may have modifications at 2′ position, for example, 2′-O-methyl RNA residue incorporation to retain full potency compared with unmodified RNA, retaining stability in serum and significantly reducing the risk of potential IFN responses in the cell. siRNA may also have 2′-fluoro modification, which is usually incorporated selectively at pyrimidine bases, to improve stability and potency.

siRNA and miRNA used as mediators of RNAi may be used as targets in, but not limited to, various infectious diseases, autoimmune/allergic diseases, heart diseases, metabolic disorders, solid tumors/cancers, hematological disorders/cancers.

In some embodiments of the present disclosure, the polynucleotide in the composition may be a polynucleotide for use in RNAi, including, without limitation, an siRNA, an miRNA, small hairpin RNA (shRNA), a long dsRNA for cleavage by Dicer, or a DsiRNA, all as described above.

In an embodiment, the negatively charged molecule may be an antagomir. Antagomirs (also known as anti-miRs or blockmirs) are synthetically engineered oligonucleotides that silence endogenous miRNA. It is unclear how antagomirization (the process by which an antagomir inhibits miRNA activity) operates, but it is believed to inhibit by irreversibly binding the miRNA. Because of the promiscuity of microRNAs, antagomirs could affect the regulation of many different mRNA molecules. Antagomirs are designed to have a sequence that is complementary to an mRNA sequence that serves as a binding site for microRNA.

In an embodiment. the negatively charged molecule may be a catalytic DNA (deoxyribozyne) or a catalytic RNA (ribozyme). As used herein, the term “catalytic DNA” refers to any DNA molecule with enzymnatic activity. In an embodiment, the catalyuic DNA is a single-stranded DNA molecule. In an embodiment, the catalytic DNA is synthetically produced as opposed to naturally occurring.

The catalytic DNA may perform one or more chemical reactions. In an embodiment, the catalytic DNA is a ribonuclease. whereby the catalytic DNA catalyzes the cleavage of ribonucleotide phosphodiester bonds. In another embodiment, the catalytic DNA is a DNA ligase, % hereby the catalytic DNA catalyzes the joining of two polynucleotide molecules by forming a new bond. In other embodiments, the catalytic DNA can catalyze DNA phosphorylation, DNA adenylation, DNA deglycosylation, porphyrin metalation, thymine dimer photoreversion, or DNA cleavage.

As used herein, the term “catalytic RNA” refers to any RNA molecule with enzymatic activity. Catalytic RNAs are involved in a number of biological processes, including RNA processing and protein synthesis. In an embodiment, the catalytic RNA is a naturally occurring RNA. In an embodiment, the catalytic RNA is synthetically produced.

The subject may be any subject to which it is desired to deliver a polynucleotide. The subject is preferably a vertebrate, such as a bird, fish or mammal, preferably a human.

The polynucleotide may delivered in various forms. In some embodiments, a naked polynucleotide may be used, either in linear form, or inserted into a plasmid, such as an expression plasmid. In other embodiments, a live vector such as a viral or bacterial vector may be used.

Depending on the nature of the polynucleotide and the intended use, one or more regulatory sequences that aid in transcription of DNA into RNA and/or translation of RNA into a polypeptide may be present. For example, if it is intended or not required that the polynucleotide be transcribed or translated, such regulatory sequences may be absent. In some instances, such as in the case of a polynucleotide that is a messenger RNA (mRNA) molecule, regulatory sequences relating to the transcription process (e.g., a promoter) are not required, and protein expression may be effected in the absence of a promoter. The skilled artisan can include suitable regulatory sequences as the circumstances require.

In some embodiments, the polynucleotide is present in an expression cassette, in which it is operably linked to regulatory sequences that will permit the polynucleotide to be expressed in the subject to which the composition of the disclosure is administered. The choice of expression cassette depends on the subject to which the composition is administered as well as the features desired for the expressed polypeptide.

Typically, an expression cassette includes a promoter that is functional in the subject and can be constitutive or inducible; a ribosome binding site; a start codon (ATG) if necessary; the polynucleotide encoding the polypeptide of interest; a stop codon; and optionally a 3′ terminal region (translation and/or transcription terminator). Additional sequences such as a region encoding a signal peptide may be included. The polynucleotide encoding the polypeptide of interest may be homologous or heterologous to any of the other regulatory sequences in the expression cassette. Sequences to be expressed together with the polypeptide of interest, such as a signal peptide encoding region, are typically located adjacent to the polynucleotide encoding the protein to be expressed and placed in proper reading frame. The open reading frame constituted by the polynucleotide encoding the protein to be expressed solely or together with any other sequence to be expressed (e.g., the signal peptide), is placed under the control of the promoter so that transcription and translation occur in the subject to which the composition is administered.

Promoters suitable for expression of polynucleotides in a wide range of host systems are well-known in the art. Promoters suitable for expression of polynucleotides in mammals include those that function constitutively, ubiquitously or tissue-specifically. Examples of non-tissue specific promoters include promoters of viral origin. Examples of viral promoters include Mouse Mammary Tumor Virus (MMTV) promoter, Human Immunodeficiency Virus Long Terminal Repeat (HIV LTR) promoter, Moloney virus, avian leukosis virus (ALV), Cytomegalovirus (CMV) immediate early promoter/enhancer, Rous Sarcoma Virus (RSV), adeno-associated virus (AAV) promoters; adenoviral promoters, and Epstein Barr Virus (EBV) promoters. Compatibility of viral promoters with certain polypeptides is a consideration since their combination may affect expression levels. It is possible to use synthetic promoter/enhancers to optimize expression (see e.g., US patent publication 2004/0171573, which is hereby incorporated by reference in its entirety).

An example of a tissue-specific promoter is the desmin promoter which drives expression in muscle cells (Li et al. 1989, Gene 78:243; Li & Paulin 1991, J. Biol. Chem. 266:6562 and Li & Paulin 1993, J. Biol. Chem. 268:10403, which are hereby incorporated by reference in their entireties). Other examples include artificial promoters such as a synthetic muscle specific promoter and a chimeric muscle-specific/CMV promoter (Li et al. 1999, Nat. Biotechnol. 17:241-245; Hagstrom et al. 2000, Blood 95:2536-2542, which are hereby incorporated by reference in their entireties).

Useful vectors are described innumerous publications, specifically WO 94/21797 and Hartikka et al. 1996, Human Gene Therapy 7:1205, which are hereby incorporated by reference in their entireties.

As noted above, the polynucleotide of interest, together with any necessary regulatory sequences, may be delivered naked, e.g., either alone or as part of a plasmid, or may be delivered in a viral or bacterial or bacterial vector.

Whether a plasmid-type vector, or a bacterial or viral vector is used, it may be desirable that the vector be unable to replicate or integrate substantially in the subject. Such vectors include those whose sequences are free of regions of substantial identity to the genome of the subject, as to minimize the risk of host-vector recombination. One way to do this is to use promoters not derived from the recipient genome to drive expression of the polypeptide of interest. For example, if the recipient is a mammal, the promoter is preferably non-mammalian derived though it should be able to function in mammalian cells, e.g., a viral promoter.

Viral vectors that may be used to deliver the polynucleotide include e.g., adenoviruses, lentiviruses and poxviruses. Useful bacterial vectors include e.g., Shigella, Salmonella, Vibrio cholerae, Lactobacillus, Bacille bilie de Calmette-Guerin (BCG), and Streptococcus.

An example of an adenovirus vector, as well as a method for constructing an adenovirus vector capable of expressing a polynucleotide, is described in U.S. Pat. No. 4,920,209, which is hereby incorporated by reference in its entirety. Poxvirus vectors include vaccinia and canary pox virus, described in U.S. Pat. Nos. 4,722,848 and 5,364,773 (which are hereby incorporated by reference in their entireties), respectively. Also see, e.g., Tartaglia et al. 1992, Virology 188:217 (which is hereby incorporated by reference in its entirety) for a description of a vaccinia virus vector and Taylor et al. 1995, Vaccine 13:539 (which is hereby incorporated by reference in its entirety) for a reference of a canary pox. Poxvirus vectors capable of expressing a polynucleotide of interest may be obtained by homologous recombination as described in Kieny et al. 1984, Nature 312:163 (which is hereby incorporated by reference in its entirety), so that the polynucleotide is inserted in the viral genome under appropriate conditions for expression in mammalian cells.

With respect to bacterial vectors, non-toxicogenic Vibrio cholerae mutant strains that are useful for expressing a foreign polynucleotide in a host are known. Mekalanos et al. 1983, Nature 306:551 and U.S. Pat. No. 4,882,278 (which are hereby incorporated by reference in their entireties) describe strains which have a substantial amount of the coding sequence of each of the two ctxA alleles deleted so that no functional cholerae toxin is produced. WO 92/11354 (which is hereby incorporated by reference in its entirety) describes a strain in which the irgA locus is inactivated by mutation; this mutation can be combined in a single strain with ctxA mutations. WO 94/01533 (which is hereby incorporated by reference in its entirety) describes a deletion mutant lacking functional ctxA and attRS1 DNA sequences. These mutant strains are genetically engineered to express heterologous proteins, as described in WO 94/19482 (which is hereby incorporated by reference in its entirety).

Attenuated Salmonella typhimurium strains, genetically engineered for recombinant expression of heterologous proteins are described in Nakayama et al. 1988, Bio/Technology 6:693 and WO 92/11361, which are hereby incorporated by reference in their entireties.

Other bacterial strains which may be used as vectors to express a foreign protein in a subject are described for Shigella flexneri in High et al. 1992, EMBO 11:1991 and Sizemore et al. 1995, Science 270:299; for Streptococcus gordonii in Medaglini et al. 1995, Proc. Natl. Acad. Sci. USA. 92:6868; and for Bacille Calmette Guerin in Flynn 1994, Cell. Mol. Biol. 40 (suppl. I):31, WO 88/06626, WO 90/00594, WO 91/13157, WO 92/01796, and WO 92/21376, all of which are hereby incorporated by reference in their entireties.

In bacterial vectors, the polynucleotide of interest may be inserted into the bacterial genome or remain in a free state as part of a plasmid.

In some embodiments, the negatively charged molecule (e.g., polynucleotide) may be present in the composition of the present disclosure at about 0.01-2 mg/mL. In some embodiments, the negatively charged molecule (e.g., polynucleotide) may be present in the composition of the present disclosure at about 0.01 mg/mL, 0.02 mg/mL, 0.03 mg/mL, 0.04 mg/mL 0.05 mg/mL, 0.06 mg/mL, 0.07 mg/mL, 0.08 mg/mL, 0.09 mg/mL, 0.1 mg/mL, 0.11 mg/mL, 0.12 mg/mL, 0.13 mg/mL, 0.14 mg/mL 0.15 mg/mL, 0.16 mg/mL, 0.17 mg/mL, 0.18 mg/mL, 0.19 mg/mL, 0.2 mg/ml, 0.25 mg/mL, 0.3 mg/mL, 0.35 mg/mL, 0.4 mg/mL, 0.45 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, or 2 mg/mL. In some embodiments, the negatively charged molecule (e.g., polynucleotide) may be present in the composition of the present disclosure at about 0.01-0.1 mg/mL, 0.05-0.2 mg/mL, 0.1-0.3 mg/mL, 0.2-0.4 mg/mL, 0.3-0.6 mg/mL, 0.4-0.8 mg/mL, 0.5-1 mg/mL, 0.8-1.2 mg/mL, 1-1.5 mg/mL, or 1-2 mg/mL. In one embodiment, the negatively charged molecule (e.g., polynucleotide) may be present in the composition of the present disclosure at about 0.1 mg/mL.

In an embodiment, the composition disclosed herein comprise a single type of negatively charged molecule in a composition. In another embodiment, the composition disclosed herein comprise a mixture of multiple different negatively charged molecules in a single composition. In an embodiment, the composition disclosed herein comprise a mixture of 2, 3,4, 5, 6, 7, 8, 9, 10, 11, 12, 13, 14, 15, 16, 17, 18, 19, 20, 21, 22, 23, 24, 25, 26, 27, 28, 29, 30 or more different negatively charged molecules in a single composition.

### Lipids

Any lipid may be used in the composition described herein so long as it is a membrane-forming lipid.

Although any lipid as defined above may be used, particularly suitable lipids may include those with at least one fatty acid chain containing at least 4 carbons, and typically about 4 to 28 carbons. The fatty acid chain may contain any number of saturated and/or unsaturated bonds. The lipid may be a natural lipid or a synthetic lipid. Non-limiting examples of lipids may include phospholipids, sphingolipids, sphingomyelin, cerobrocides, gangliosides, ether lipids, sterols, cardiolipin, cationic lipids and lipids modified with poly (ethylene glycol) and other polymers. Synthetic lipids may include, without limitation, the following fatty acid constituents: lauroyl, myristoyl, palmitoyl, stearoyl, arachidoyl, oleoyl, linoleoyl, erucoyl, or combinations of these fatty acids. In some embodiments, the lipid or lipids of the lipid vesicle particle are amphiphilic lipids, meaning that they possess both hydrophilic and hydrophobic (lipophilic) properties.

Lipids suitable for use in the composition of the present disclosure include, but are not limited to phospholipids, cationic lipids, cholesterol and/or cholesterol derivatives, or a combination thereof. It is to be understood that the terms “phospholipids”, “cationic lipids” or “cholesterol derivatives”, are not necessarily mutually exclusive of each other.

Broadly defined, a “phospholipid” is a member of a group of lipid compounds that yield on hydrolysis phosphoric acid, an alcohol, fatty acid, and nitrogenous base. Phospholipids that are preferably used in the preparation of the composition of the present disclosure are those with at least one head group selected from the group consisting of phosphoglycerol, phosphoethanolamine, phosphoserine, phosphocholine and phosphoinositol. More preferred are lipids which are about 94-100% phosphatidylcholine. Such lipids are available commercially in the lecithin Phospholipon® 90 G (Phospholipid GmBH, Germany) or lecithin ST00 (Lipoid GmBH, Germany). In some embodiments, the phospholipid used in the preparation of the composition of the present disclosure is dioleoyl phosphatidylcholine (DOPC), 1,2-dipalmitoyl-sn-glycero-3-phosphocholine (DPPC), Dioleoyl Phosphatidylethanolamine (DOPE), 1,2-dipalmitoyl-sn-glycero-3-succinate (DGS), or a combination thereof. In one embodiment, the phospholipid used in the preparation of the composition of the present disclosure is dioleoyl phosphatidylcholine (DOPC). In some embodiments, a mixture of DOPC and unesterified cholesterol may be used. In other embodiments, a mixture of Lipoid S 100 lecithin and unesterified cholesterol may be used.

In one embodiment, the lipid vesicle particles comprise a synthetic lipid. In an embodiment, the lipid vesicle particles comprise synthetic DOPC. In another embodiment, the lipid vesicle particles comprise synthetic DOPC and cholesterol.

Another common phospholipid is sphingomyelin. Sphingomyelin contains sphingosine, an amino alcohol with a long unsaturated hydrocarbon chain. A fatty acyl side chain is linked to the amino group of sphingosine by an amide bond, to form ceramide. The hydroxyl group of sphingosine is esterified to phosphocholine. Like phosphoglycerides, sphingomyelin is amphipathic.

Lecithin, which also may be used, is a natural mixture of phospholipids typically derived from chicken eggs, sheep's wool, soybean and other vegetable sources.

All of these and other phospholipids may be used in the practice of the disclosure. Phospholipids can be purchased, for example, from Avanti lipids (Alabastar, AL, USA), Lipoid LLC (Newark, NJ, USA) and Lipoid GmbH (Germany), among various other suppliers.

Cholesterol and/or cholesterol derivatives may be used in the composition of the present disclosure. When unesterified cholesterol is used in the composition, the cholesterol is usually used in an amount equivalent to about 10% of the amount of phospholipid. If a compound other than cholesterol is used to stabilize the composition, one skilled in the art can readily determine the amount needed in the composition. Cholesterol derivatives suitable for use in the present disclosure include cholesterol β-D-glucoside, cholesterol 3-sulfate sodium salt, positively charged cholesterol such as DC-cholesterol and other cholesterol like molecules such as Campesterol, Ergosterol, Betulin, Lupeol, β-Sitosterol, α,β-Amyrin and bile acids.

In some embodiments, the lipid vesicle particles comprise DOPC and cholesterol at a DOPC:Cholesterol ratio of about 10:1 (w/w). In some embodiments, the lipid vesicle particles comprise DOPC and cholesterol at a DOPC:cholesterol ratio of about 8:1 (w/w), about 9:1 (w/w), about 11:1 (w/w), or about 12:1 (w/w).

In one embodiment, the compositions disclosed herein comprise about 66 mg/ml of DOPC and cholesterol. In other embodiments, the compositions disclosed herein comprise about 55 mg/ml, 56 mg/ml, 57 mg/ml, 58 mg/ml, 59 mg/ml, 60 mg/ml, 61 mg/ml, 62 mg/ml, 63 mg/ml, 64 mg/ml, 65 mg/ml, 67 mg/ml, 68 mg/ml, 69 mg/ml, 70 mg/ml, 71 mg/ml, 72 mg/ml, 73 mg/ml, 74 mg/ml, or 75 mg/ml of DOPC and cholesterol.

In one embodiment, the compositions disclosed herein comprise about 60 mg/ml of DOPC and about 6 mg/ml of cholesterol.

In some embodiments, positively charged lipids (or cationic lipids) are used in the composition of the present disclosure. Exemplary cationic lipids suitable for use in the compositions of the present disclosure include but are not limited to, 1,2-dioleoyl-3-trimethylammonium-propane (DOTAP), 1-[2-(oleoyloxy)ethyl]-2-oleyl-3-(2-hydroxyethyl)imidazolinium chloride (DOTIM), N-[1-(2,3-dioleyloxy)propyl]-N,N,N-trimethylammonium chloride (DOTMA), dioctadecylamidoglycylspermine-4trifluoroacetic acid (DOGS), dioleyldimethylammonium chloride (DODAC), dimethyldioctadecylammonium bromide (DDAB), 1,2-distearoyl-3-dimethylammonium-propane (DAP), N-(4-carboxybenzyl)-N,N-dimethyl-2,3-bis(oleoyloxy)propan-1-aminium (DOBAQ), 1,2-dipalmitoyl-sn-glycero-3-succinate (DGS), N-palmitoyl homocysteine ammonium salt (PHC), 1,2-dioleyloxy-3-dimethylaminopropane (DODMA), Dimethyldioctadecylammonium Bromide Salt (DDAB), 1,2-dilauroyl-sn-glycero-3-ethylphosphocholine chloride salt (EPC), N4-Cholesteryl-Spermine HCl Salt (GL67), 1,2-dioleoyloxypropyl-3-dimethyl-hydroxyethylammonium bromide (DORI), N-(3-aminopropyl)-N,N-dimethyl-2,3-bis(dodecyloxy)-1-propanammonium bromide (GAP-DLRIE), 2,3dioleyloxy-N-[2[sperminecarboxaminino]ethyl]-N,N-dimethyl-1-propanaminium trifluroacetate (DOSPA), 1,2-dimyristyloxypropyl-3-dimethyl-hydroxy ethyl ammonium bromide (DMRIE), 1,2-distearoyl-sn-glycero-3-phosphoethanolamine (DSPE), and SAINT 2. Further examples of cationic lipids include those described in, for example, Audouy and Hoekstra, Mol Membr Biol, April-June 2001; 18(2):129-43; Shim et al., Asian Journal of Pharmaceutical Sciences 8(2):72-80, April 2013; and Faneca et al (2013) Cationic Liposome-Based Systems for Nucleic Acid Delivery: From the Formulation Development to Therapeutic Applications. In: Coelho J. (eds) Drug Delivery Systems: Advanced Technologies Potentially Applicable in Personalised Treatment. Advances in Predictive, Preventive and Personalised Medicine, vol 4. Springer, Dordrecht, which are incorporated herein by reference in their entireties.

The lipid vesicle particles may have closed vesicular structures. They are typically spherical in shape, but other shapes and conformations may be formed and are not excluded. Exemplary embodiments of lipid vesicle particles include, without limitation, single layer vesicular structures (e.g., micelles) and bilayer vesicular structures (e.g., unilamellar or multilamellar vesicles), or various combinations thereof.

By “single layer” it is meant that the lipids do not form a bilayer, but rather remain in a layer with the hydrophobic part oriented on one side and the hydrophilic part oriented on the opposite side. By “bilayer” it is meant that the lipids form a two-layered sheet, typically with the hydrophobic part of each layer internally oriented toward the center of the bilayer with the hydrophilic part externally oriented. However, the opposite configuration is also possible. The term “multilayer” is meant to encompass any combination of single and bilayer structures. The form adopted may depend upon the specific lipid that is used.

In an embodiment, the lipid vesicle particle is a bilayer vesicular structure, such as for example, a liposome. Liposomes are completely closed lipid bilayer membranes. Liposomes may be unilamellar vesicles (possessing a single bilayer membrane), multilamellar vesicles (characterized by multimembrane bilayers whereby each bilayer may or may not be separated from the next by an aqueous layer) or multivesicular vesicles (possessing one or more vesicles within a vesicle). A general discussion of liposomes can be found in Gregoriadis 1990; and Frezard 1999, which are incorporated herein by reference in their entirety.

Thus, in an embodiment, the lipid vesicle particles are liposomes. In an embodiment, the liposomes are unilamellar, multilamellar, multivesicular or a mixture thereof.

### Carriers

In some embodiments, the carrier of the composition comprises a continuous phase of a hydrophobic substance, preferably a liquid hydrophobic substance. The continuous phase may be an essentially pure hydrophobic substance or a mixture of hydrophobic substances. In addition, the carrier may be an emulsion of water in a hydrophobic substance or an emulsion of water in a mixture of hydrophobic substances, provided the hydrophobic substance constitutes the continuous phase. Further, in another embodiment, the carrier may function as an adjuvant.

Hydrophobic substances that are useful in the compositions as described herein are those that are pharmaceutically and/or immunologically acceptable. The carrier is preferably a liquid but certain hydrophobic substances that are not liquids at atmospheric temperature may be liquefied, for example by warming, and are also useful in this disclosure. In one embodiment, the hydrophobic carrier may be a PBS/FIA emulsion.

Oil or water-in-oil emulsions are particularly suitable carriers for use in the present disclosure. Oils should be pharmaceutically and/or immunologically acceptable. Suitable oils include, for example, mineral oils (especially light or low viscosity mineral oil such as Drakeo® 6VR), vegetable oils (e.g., soybean oil), nut oils (e.g., peanut oil), or mixtures thereof. In an embodiment, the oil is a mannide oleate in mineral oil solution, commercially available as Montanide® ISA 51. In one embodiment, the oil is MS80 oil (mixture of mineral oil and Span 80). Animal fats and artificial hydrophobic polymeric materials, particularly those that are liquid at atmospheric temperature or that can be liquefied relatively easily, may also be used. Mixtures of different hydrophobic substances, such as mixtures that include one or more different oils, animal fats or artificial hydrophobic polymeric materials, may be used.

### Ionizable Aminoglycoside

In some aspects, composition of the present disclosure contains an ionizable aminoglycoside. Ionizable aminoglycosides useful in the compositions of the present disclosure include, but are not limited to, chitosan, cationic alginate, cationic gelatin, cationic dextran, DEAE-dextran hydrochloride, aminated cellulose, aminated sucrose, aminated trehalose, N-acetyl-D-glucosamine, D-(+)-glucosamine hydrochloride, glycyrrhizic acid ammonium salt, or derivatives thereof.

In one embodiment, the ionizable aminoglycoside is a polymer. In one embodiment, the ionizable aminoglycoside is chitosan, chitosan derivative, or a chitosan like molecule. Chitosan derivatives suitable for use in the present disclosure include, but are not limited to N-trimethyl chitosan, mono-N-carboxymethyl chitosan (MCC), N-palmitoyl chitosan (NPCS), EDTA-chitosan, low molecular weight chitosan, galactosylated chitosan, N-dodecylated chitosan, thiolated chitosan or combinations thereof. The chitosan like molecules include, but are not limited to, N-acetyl-D-glucosamine, D-(+)-glucosamine hydrochloride, galactosamine, N-acetylgalactosamine, cellulose acetate, mannosamine, N-acetylneuraminic acid, alginic acid, Trehalose-6,6-dibehenate (TDB) with Dimethyldioctadecylammonium bromide (DDA), heptakis(6-deoxy-6-amino)-β-cyclodextrin heptahydrochloride, DEAE-dextran hydrochloride, and glycyrrhizic acid ammonium salt. It is contemplated that the chitosan, chitosan derivative, or a chitosan like molecule may also function as a carrier (e.g., a structural carrier).

In some embodiments, the chitosan or chitosan derivative used in the composition has a molecular weight of about 10 kDa to 200 kDa. In some embodiments, the chitosan or chitosan derivative used in the composition has a molecular weight of about 60 kDa to 150 kDa, about 80 kDa to 150 kDa, about 90 kDa to 110 kDa, about 100 kDa to 120 kDa, about 100 kDa, or about 120 kDa. In some embodiments, the chitosan or chitosan derivative used in the composition has a molecular weight of about 80 kDa, about 90 kDa, about 100 kDa, about 110 kDa, about 120 kDa, about 130 kDa, about 140 kDa, or about 150 kDa. In one embodiment, the chitosan or chitosan derivative used in the composition has a molecular weight of about 100 kDa.

Degree of deacetylation (DD) is another main parameter characterizing chitosan. The degree of deacetylation (DD, %) is defined as the molar fraction of D-glusoamine units in the copolymers (chitosan) composed of N-acetylglucosamine units and D-glusoamine units (Shigemasa Y et al., Int. J. Biol. Macromol. 1996; 18:237-242, which is incorporated hereby by reference in its entirety). In some embodiments, the chitosan or chitosan derivative used in the composition has a degree of deacetylation (DD) of about 15% to 95%. In some embodiments, the chitosan or chitosan derivative used in the composition has a degree of deacetylation (DD) of about 15% to 30%, about 20% to 30%, about 20% to 40%, about 25% to 50%, about 30% to 60%, about 40% to 70%, about 50% to 80%, about 60% to 90%, about 70% to 95%. In some embodiments, the chitosan or chitosan derivative used in the composition has a degree of deacetylation (DD) of about 15%, 20%, 25%, 30%, 35%, 40%, 45%, 50%, 55%, 60%, 65%, 70%, 75%, 80%, 85%, 90%, or 95%. In one embodiment, the chitosan or chitosan derivative has a DD % of about 25%.

In some embodiments, the chitosan or chitosan derivative is added in a concentration of about 0.1 mg/mL to about 5 mg/mL. In some embodiments, the chitosan or chitosan derivative is added in a concentration of about 0.25 mg/mL to about 4 mg/mL, about 0.5 mg/mL to about 3 mg/mL, about 0.75 mg/mL to about 2.5 mg/mL, about 1 mg/mL to about 2 mg/mL. In some embodiments, the chitosan or chitosan derivative is added in a concentration of about 0.5 mg/mL to about 3 mg/mL or about 1 mg/mL to about 2 mg/mL. In some embodiments, the chitosan or chitosan derivative is added in a concentration of about 0.1 mg/ml, about 0.25 mg/ml, about 0.5 mg/ml, about 0.75 mg/ml, about 1 mg/mL, about 1.25 mg/ml, about 1.5 mg/mL, about 1.75 mg/ml, about 2 mg/mL, about 2.25 mg/ml, about 2.5 mg/ml, about 2.75 mg/ml, about 3 mg/ml, about 3.25 mg/ml, about 3.5 mg/ml, about 3.75 mg/ml, about 4 mg/ml, about 4.25 mg/ml, about 4.5 mg/ml, about 4.75 mg/ml, about 5 mg/ml. In some embodiments, the chitosan or chitosan derivative is added in a concentration of about 0.1 mg/ml, about 1.5 mg/mL, about 2 mg/mL, about 2.5 mg/mL, or about 3 mg/mL.

### Adjuvants

The composition may comprise one or more adjuvants. For example, if the encoded polypeptide is a vaccine antigen, an adjuvant may be present. An adjuvant can serve as a tissue depot that slowly releases the antigen and also as a lymphoid system activator that non-specifically enhances the immune response (Hood et al, Immunology, 2d ed., Benjamin/Cummings: Menlo Park, C. A., 1984; see Wood and Williams, In: Nicholson, Webster and May (eds.), Textbook of Influenza, Chapter 23, pp. 317-323, which are incorporated hereby by reference in their entireties). Often, a primary challenge with an antigen alone, in the absence of an adjuvant, will fail to elicit an adaptive immune response. It should be noted that the polynucleotide of interest to be delivered to the subject may itself function as an adjuvant, or may encode a polypeptide that constitutes an adjuvant (e.g., IL-12, IFN-gamma, or Granulocyte-Macrophage Colony Stimulating Factor (“GMCSF”)).

In some embodiments, the adjuvant is a protein, a polymer, a polysaccharide, or a combination thereof. The adjuvant may be a natural or synthetic substance.

The amount of adjuvant used depends on the amount of antigen expressed by the polynucleotide and on the type of adjuvant. One skilled in the art can readily determine the amount of adjuvant needed in a particular application.

Suitable adjuvants include, but are not limited to, alum, other compounds of aluminum, Bacillus of Calmette and Guerin (BCG), TiterMax®, incomplete Freund's adjuvant (IFA), saponin, surface active substances such as lysolecithin, pluronic polyols, polyanions, peptides, Corynebacterium parvum, QS-21, and Freund's Complete Adjuvant (FCA), adjuvants of the STING family Bis-(3′-5′)-cyclic dimeric guanosine monophosphate (c-di-GMP), Bis-(3′-5′)-cyclic dimeric adenosine monophosphate (c-di-AMP) and cyclic di-inosine monophosphate (c-di-IMP), adjuvants of the TLR agonist family such as CpG-containing oligodeoxynucleotides (CpG ODN), polyL:C, falgellin, lipopeptides, peptidoglycans, lipid-based adjuvant (e.g., palmitic acid adjuvant), imidazoquinolines, single stranded RNA, lipopolysaccharides (LPS), heat shock proteins (HSP), cationic albumin, and ceramides and derivatives such as alpha Gal-cer. Suitable adjuvants also include cytokines or chemokines in their polypeptide or DNA coding forms such as, but not limited to, GM-CSF, TNF-alpha, IFN-gamma, IL-2, IL-12, IL-15, IL-21. Suitable adjuvants that activate or increases the activity of TLR2 include those described in U.S. Pat. Nos. 10,105,435 and 10,022,441, which are hereby incorporated by reference in their entirety.

In an embodiment, the adjuvant is a CpG ODN. CpG ODNs are DNA molecules that contain one or more unmethylated CpG motifs (consisting of a central unmethylated CG dinucleotide plus flanking regions). An exemplary CpG ODN is 5′-TCCATGACGTTCCTGACGTT-3′ (SEQ ID NO: 1). The skilled person can readily select other appropriate CpG ODNs on the basis of the target species and efficacy.

In an embodiment, the adjuvant may be a polyL:C polynucleotide. PolyI:C polynucleotides are polynucleotide molecules (either RNA or DNA or a combination of DNA and RNA) containing inosinic acid residues (I) and cytidylic acid residues (C), and which induce the production of inflammatory cytokines, such as interferon. In an embodiment, the polyL:C polynucleotide is double-stranded. In such embodiments, they may be composed of one strand consisting entirely of cytosine-containing nucleotides and one strand consisting entirely of inosine-containing nucleotides, although other configurations are possible. For instance, each strand may contain both cytosine-containing and inosine-containing nucleotides. In some instances, either or both strands may additionally contain one or more non-cytosine or non-inosine nucleotides.

It has been reported that polyL:C can be segmented every 16 residues without an effect on its interferon activating potential (Bobst 1981). Furthermore, the interferon inducing potential of a polyL:C molecule mismatched by introducing a uridine residue every 12 repeating cytidylic acid residues (Hendrix 1993), suggests that a minimal double stranded polyL:C molecule of 12 residues is sufficient to promote interferon production. Others have also suggested that regions as small as 6-12 residues, which correspond to 0.5-1 helical turn of the double stranded polynucleotide, are capable of triggering the induction process (Greene 1978). If synthetically made, polyL:C polynucleotides are typically about 20 or more residues in length (commonly 22, 24, 26, 28 or 30 residues in length). If semi-synthetically made (e.g. using an enzyme), the length of the strand may be 500, 1000 or more residues.

Accordingly, as used herein, a “polyL:C”, “polyL:C polynucleotide” or “polyL:C polynucleotide adjuvant” is a double- or single-stranded polynucleotide molecule (RNA or DNA or a combination of DNA and RNA), each strand of which contains at least 6 contiguous inosinic or cytidylic acid residues, or 6 contiguous residues selected from inosinic acid and cytidylic acid in any order (e.g., IICIIC or ICICIC), and which is capable of inducing or enhancing the production of at least one inflammatory cytokine, such as interferon, in a mammalian subject. PolyI:C polynucleotides will typically have a length of about 8, 10, 12, 14, 16, 18, 20, 22, 24, 25, 26, 28, 30, 35, 40, 45, 50, 55, 60, 65, 70, 75, 80, 85, 90, 95, 100, 150, 200, 250, 300, 500, 1000 or more residues. Preferred polyL:C polynucleotides may have a minimum length of about 6, 8, 10, 12, 14, 16, 18, 20, 22, 24, 26, 28, or 30 nucleotides and a maximum length of about 1000, 500, 300, 200, 100, 90, 80, 70, 60, 50, 45 or 40 nucleotides.

Each strand of a double-stranded polyL:C polynucleotide may be a homopolymer of inosinic or cytidylic acid residues, or each strand may be a heteropolymer containing both inosinic and cytidylic acid residues. In either case, the polymer may be interrupted by one or more non-inosinic or non-cytidylic acid residues (e.g. uridine), provided there is at least one contiguous region of 6 I, 6 C or 6 I/C residues as described above. Typically, each strand of a polyL:C polynucleotide will contain no more than 1 non-I/C residue per 6 I/C residues, more preferably, no more than 1 non-I/C residue per every 8, 10, 12, 14, 16, 18, 20, 22, 24, 26, 28 or 30 I/C residues.

The inosinic acid or cytidylic acid (or other) residues in the polyL:C polynucleotide may be derivatized or modified as is known in the art, provided the ability of the polyL:C polynucleotide to promote the production of an inflammatory cytokine, such as interferon, is retained. Non-limiting examples of derivatives or modifications include e.g. azido modifications, fluoro modifications, or the use of thioester (or similar) linkages instead of natural phosphodiester linkages to enhance stability in vivo. The polyL:C polynucleotide may also be modified to e.g. enhance its resistance to degradation in vivo by e.g. complexing the molecule with positively charged poly-lysine and carboxymethylcellulose, or with a positively charged synthetic peptide.

In an embodiment, the polyL:C polynucleotide may be a single-stranded molecule containing inosinic acid residues (I) and cytidylic acid residues (C). As an example, and without limitation, the single-stranded polyL:C may be a sequence of repeating dIdC. In a particular embodiment, the sequence of the single-stranded polyL:C may be a 26-mer sequence of (IC)₁₃, i.e. ICICICICICICICICICICICICIC (SEQ ID NO: 2). As the skilled person will appreciate, due to their nature (e.g., complementarity), it is anticipated that these single-stranded molecules of repeating dIdC would naturally form homodimers, so they are conceptually similar to polyI/polyC dimers.

In an embodiment, the polyL:C polynucleotide adjuvant is a traditional form of polyL:C with an approximate molecular weight of 989,486 Daltons, containing a mixture of varying strand lengths of polyI and polyC of several hundred base pairs (Thermo Scientific; USA).

In an embodiment, the adjuvant may be one that activates or increases the activity of TLR2. As used herein, an adjuvant which “activates” or “increases the activity” of a TLR2 includes any adjuvant, in some embodiments a lipid-based adjuvant, which acts as a TLR2 agonist. Further, activating or increasing the activity of TLR2 encompasses its activation in any monomeric, homodimeric or heterodimeric form, and particularly includes the activation of TLR2 as a heterodimer with TLR1 or TLR6 (i.e. TLR1/2 or TLR2/6). Exemplary embodiments of an adjuvant that activates or increases the activity of TLR2 include lipid-based adjuvants, such as those described in WO2013/049941, which is hereby incorporated by reference in its entirety.

In an embodiment, the adjuvant may be a lipid-based adjuvant, such as disclosed for example in WO2013/049941, which is hereby incorporated by reference in its entirety. In an embodiment, the lipid-based adjuvant is one that comprises a palmitic acid moiety such as dipalmitoyl-S-glyceryl-cysteine (PAM2Cys) or tripalmitoyl-S-glyceryl-cysteine (PAM3Cys). In an embodiment, the adjuvant is a lipopeptide. Exemplary lipopeptides include, without limitation, PAM2Cys-Ser-(Lys)4 (SEQ ID NO: 3) or PAM3Cys-Ser-(Lys)4 (SEQ ID NO: 4).

In an embodiment, the adjuvant is PAM3Cys-SKKKK (EMC Microcollections, Germany; SEQ ID NO: 5) or a variant, homolog and analog thereof. The PAM2 family of lipopeptides has been shown to be an effective alternative to the PAM3 family of lipopeptides.

In an embodiment, the adjuvant may be a lipid A mimic or analog adjuvant, such as for example those disclosed in WO2016/109880 and the references cited therein, which are hereby incorporated by reference in their entireties. In a particular embodiment, the adjuvant may be JL-265 or JL-266 as disclosed in WO2016/109880, which is hereby incorporated by reference in its entirety.

In an embodiment, a combination of a polyL:C polynucleotide adjuvant and a lipid-based adjuvant may be used, such as described in the adjuvanting system disclosed in WO2017/083963, which is hereby incorporated by reference in its entirety.

Further examples of compatible adjuvants may include, without limitation, chemokines, Toll like receptor agonists, colony stimulating factors, cytokines, 1018 ISS, aluminum salts, Amplivax, ASO4, AS15, ABM2, Adjumer, Algammulin, ASO1B, ASO2 (SBASA). ASO2A, BCG, Calcitriol, Cholera toxin, CP-870,893, CpG, polyIC, CyaA, Dimethyldioctadecylammonium bromide (DDA), Dibutyl phthalate (DBP), Trehalose-6,6-dibehenate (TDB), dSLIM, Gamma inulin, GM-CSF, GMDP, Glycerol, IC30, IC31, Imiquimod, ImuFact IMP321. IS Patch, ISCOM, ISCOMATRIX, JuvImmune, LipoVac, LPS, lipid core protein, MF59, monophosphoryl lipid A, Montanide® IMS1312, Montanide® based adjuvants, OK-432, OM-174, OM-197-MP-EC, ONTAK, PepTel vector system, other palmitoyl based molecules, PLG microparticles, resiquimod, squalene, SLR172, YF-17 DBCG, QS21, QuilA, P1005, Poloxamer, synthetic polynucleotides, Zymosan, and pertussis toxin.

A wide range of pharmaceutically acceptable adjuvants, excipients, etc. are known in the art and may be used in the compositions of the disclosure: See, for example, Remington's Pharmaceutical Sciences (Remington's Pharmaceutical Sciences, Mack Publishing Company, Easton, Pa., USA 1985) and The United States Pharmacopoeia: The National Formulary (USP 24 NF19) published in 1999, which are incorporated hereby by reference in their entireties.

### Additional Components

The composition may further comprise one or more additional components that may facilitate the delivery of the negatively charged molecule (e.g., polynucleotide) to the subject. The additional components may enhance the stability of the composition, or complement or enhance the function of the negatively charged molecule (e.g., polynucleotide) to be delivered to the subject.

In some embodiments, the composition further comprises a buffer. A buffer works to maintain the pH of solution to prevent a sharp pH change in the liquid formulation for stabilizing the composition. The buffer may include an alkaline salt (sodium or potassium phosphate or hydrogen or dihydrogen salts thereof), sodium citrate/citric acid, sodium acetate/acetic acid, and any other pharmaceutically acceptable pH buffer known in the art, and a combination thereof. The preferred example of such buffer includes an acetate buffer, citrate buffer, and phosphate buffer. The buffer may also include zwitterionic salts such THAM tris(hydroxymethyl)aminomethane, HEPES (4-(2-hydroxyethyl)-1-piperazineethanesulfonic acid), MOPS (3-(N-Morpholino) propanesulfonic acid) and MES (2-(N-morpholino)ethanesulfonic acid).

In some embodiments, the buffer is sodium acetate. In some embodiments, the concentration of the sodium acetate buffer is about 25-250 mM. In some embodiments, the concentration of the sodium acetate buffer is about 100 mM. In some embodiments, the sodium acetate buffer has a pH of 6.0-10.5. In some embodiments, the sodium acetate buffer has a pH of about 7.

In some embodiments, the composition further comprises a surfactant. Exemplary surfactants may include, but are not limited to sorbitan monooleate, Cremophor, polysorbate 20, polysorbate 40, polysorbate 60, polysorbate 80, poloxamers, polyethylene glycol, transcutol, Capmul®, labrasol, isopropyl myristate, and/or Span 80.

If an additional component in the composition is a polypeptide, a polynucleotide encoding the additional polypeptide may instead be provided, in the same manner as for the polynucleotide encoding the polypeptide of primary interest. Such polypeptides could be expressed from the same or separate expression vectors, or could be expressed in the form of a fusion protein.

### Formulation of Compositions

The lipid vesicle particles of the present disclosure may be prepared by methods known in the art, and/or described herein, e.g., in the Examples section below.

In some embodiments, the lipid vesicle particles of the present disclosure are prepared using the methods described in International PCT Application WO/2019/090411, U.S. Pat. No. 9,498,493, which are incorporated herein by reference in their entirety.

In some embodiments, the lipid vesicle particles may form structures such as liposomes. Methods for making liposomes are well known in the art: see, for example, Gregoriadis (1990) and Frezard (1999), both cited previously. Any suitable method for making liposomes may be used in the practice of the disclosure. Liposomes are typically prepared by hydrating the liposome components that will form the lipid bilayer (e.g., phospholipids and cholesterol) with an aqueous solution, which may be pure water or any other physiologically compatible solution such as saline, e.g., phosphate-buffered saline (PBS).

In one aspect, the present disclosure provides a method for preparing a composition comprising one or more lipids, a negatively charged molecule, a carrier comprising a continuous phase of a hydrophobic substance, and an ionizable aminoglycoside. The method can comprise one or more of the following steps:
a) dissolving the one or more lipids in one or more organic solvents and optionally an aqueous solvent to create a lipid solution,
b) adding the negatively charged molecule to the lipid solution formed in step a) and mixing;
c) adding the ionizable aminoglycoside to the mixture formed in step b) and mixing;
d) optionally, adding additional amount of the organic solvent(s) or aqueous solvent to the mixture formed in step c) thereby the overall Wt/Wt or V/V percentage ratio of organic:aqueous solvent or aqueous:organic solvent in the mixture is between 20-50%;
e) drying the mixture formed in step c) or d) to generate a dried preparation; and
f) dissolving the dried preparation in the carrier comprising a continuous phase of a hydrophobic substance, thereby generating said composition.

In some embodiments, a lipid component or mixture of lipid components, such as a phospholipid (e.g., DOPC) and cholesterol, may be solubilized in one or more organic solvent, such as tert-butanol, ethanol, methanol, chloroform, or a mixture of chloroform and methanol, tert-butanol, a mixture of tert-butanol and ethanol, a mixture of tert-butanol and chloroform, or mixture of tert-butanol and water followed by filtering (e.g., a PTFE 0.2 μm filter) and drying, e.g., by rotary evaporation, freeze-drying to remove the solvents.

In some embodiments, the organic solvent(s) is present in an amount sufficient to prevent the one or more lipids from forming lipid vesicle particles in the lipid solution. In some embodiments, the organic solvent(s) and the aqueous solvent are present in a percentage ratio of organic:aqueous solvent or aqueous:organic solvent ratio between 20-50%. In some embodiments, the organic solvent(s) and the aqueous solvent are present in a percentage ratio of organic:aqueous solvent or aqueous:organic solvent ratio of about 20%, about 25%, about 30%, about 35%, about 40%, about 45%, or about 50%.

In some embodiments, the organic solvent is tert-butanol. In some embodiments, the lipid solution comprises about 20%-50% tert-butanol. In some embodiments, the lipid solution comprises about 20% tert-butanol, about 25% tert-butanol, about 30% tert-butanol, about 35% tert-butanol, about 40% tert-butanol, about 45% tert-butanol, or about 50% tert-butanol. In one embodiment, the lipid solution comprises about 30% tert-butanol.

Hydration of the resulting lipid mixture may be effected by e.g., injecting the lipid mixture into an aqueous solution or sonicating the lipid mixture and an aqueous solution. During formation of the lipid vesicle particles (e.g., liposomes), the lipid components form single bilayers (unilamellar) or multiple bilayers (multilamellar) surrounding a volume of the aqueous solution with which the lipid components are hydrated.

In some embodiments, the lipid vesicle particles are then dehydrated, such as by freeze-drying or lyophilization, spray freeze-drying, spray drying, or rotary evaporation, and subsequently reconstituted with an aqueous solution.

In some embodiments, the lipid vesicle particles are combined with the carrier comprising a continuous hydrophobic phase. In some embodiments, the hydrophobic phase is essentially water-free. This can be done in a variety of ways.

If the carrier is essentially water-free, and is composed solely of a hydrophobic substance or a mixture of hydrophobic substances (e.g., use of a 100% mineral oil carrier), lipid vesicle particles may simply be mixed with the hydrophobic substance, or if there are multiple hydrophobic substances, mixed with any one or a combination of them. An exemplary preparation method is further described below and in the Examples section (see Formulation Method A (standard). In this method, any negatively charged molecules (e.g., polynucleotide) presented in an aqueous solution (e.g., sterile RNase free water) is added to a suitable buffer solution. To this diluted polynucleotide solution, previously prepared lipid vesicle particles in sterile RNase free water (e.g., particle size <120 nm, polydispersity index (PDI)<0.1) is added, mixed well gently by e.g., hand or vortexing for 30 seconds. In some embodiments, formulation method A is prepared with different polymers and transfection agents by adding the respective stock solutions to the polynucleotide loaded lipid vesicle particles obtained from the above step, mixed well gently by e.g., hand or vortexing for 30 seconds. The final formulation (with and without different polymers and transfection agents) is then dehydrated by freeze-drying or lyophilization or spray-drying; and subsequently reconstituted with an aqueous solution or with a hydrophobic substance or a mixture of hydrophobic substances (e.g., use of a 100% mineral oil carrier) prior to administration.

If instead the carrier comprising a continuous phase of a hydrophobic substance contains a discontinuous aqueous phase, the carrier will typically take the form of an emulsion of the aqueous phase in the hydrophobic phase, such as a water-in-oil emulsion. Such compositions may contain an emulsifier to stabilize the emulsion and to promote an even distribution of the lipid vesicle particles (e.g., liposomes). In this regard, emulsifiers may be useful even if water-free carrier is used, for the purpose of promoting an even distribution of the lipid vesicle particles (e.g., liposomes) in the carrier. Typical emulsifiers include mannide oleate (Arlacel™ A), lecithin, Tween™ 80, and Spans™ 20, 80, 83 and 85. Typically, the weight to volume ratio (w/v) of hydrophobic substance to emulsifier is in the range of about 5:1 to about 15:1 with a ratio of about 10:1 being preferred.

The lipid vesicle particles may be added to the finished emulsion, or they may be present in either the aqueous phase or the hydrophobic phase prior to emulsification.

The negatively charged molecule such as polynucleotide to be expressed may be introduced at various different stages of the formulation process. In this section, the term “polynucleotide” includes the polynucleotide in naked form including, for example, in an mRNA or a plasmid such as an expression plasmid, or in a live vector such as a bacteria or virus.

An exemplary preparation method is further described below and in the Examples section (see Formulation Method B). In this method, lipid or lipid-mixture is dissolved in 20-50% or 100% tert-butanol or ethanol by, e.g., simple vortexing or by shaking at 150-300 RPM in an incubator shaker at room temperature or at 37° C. until dissolved. To the dissolved lipid solution, any negatively charged molecules (e.g., polynucleotide) presented in an aqueous solution (e.g., sterile RNase free water) or suitable buffer solution or complexed with another positively charged molecule/adjuvant with known cryoprotectants is added, mixed well gently by e.g., hand or vortexing for 30 seconds. The overall Wt/Wt or V/V percentage ratio of organic:aqueous solvent mixture aqueous:organic solvent mixture in the final formulation prior to dehydration is maintained between 20-50%. Although not wishing to be bound by theory, it is believed that the one or more lipids when dissolved in the organic solvent(s) are presented in clear solution form. When admixed or exposed to the aqueous solution(s) containing the negatively charged molecule (e.g., polynucleotide) and/or ionizable aminoglycoside, the one or more lipids form lipid vesicle particles. The formed lipid vesicle particles are then dehydrated, such as by freeze-drying or lyophilization or spray-drying; and subsequently reconstituted with an aqueous solution or with a hydrophobic substance or a mixture of hydrophobic substances (e.g., use of a 100% mineral oil carrier) prior to administration. In general, this method has several advantages over the conventional lipid nanoparticles method including increased solubility and encapsulation of hydrophobic molecules, decreased freeze-drying time, and better oil reconstitution characteristics for hydrophobic or complex compounds.

More than one polynucleotide may be incorporated into the composition. For example, two or more polynucleotides encoding different proteins may be incorporated into the composition, or a polynucleotide encoding a protein may be present as well as a polynucleotide encoding an antisense RNA or interfering RNA. Proteins may be expressed as the fusion product of two different polynucleotides. More than one polynucleotide may be under the control of the same regulatory elements, e.g., two or more polynucleotides under transcriptional control of a single promoter.

In some embodiments, the polynucleotide is present in the aqueous solution used to hydrate the components that are used to form the lipid bilayers of the lipid vesicle particles (e.g., liposomes). In this case, the polynucleotide will be encapsulated in the lipid vesicle particles, present in its aqueous interior. If the resulting liposomes are not washed or dried, such that there is residual aqueous solution present that is ultimately mixed with the carrier comprising a continuous phase of a hydrophobic substance, it is possible that additional polynucleotide may be present outside the lipid vesicle particles in the final product. In a related technique, the polynucleotide may be mixed directly with the lipid vesicle particles or with the components used to form the lipid bilayers of the lipid vesicle particles, prior to hydration with the aqueous solution.

In an alternative approach, the polynucleotide may instead be mixed with the carrier comprising a continuous phase of a hydrophobic substance, before, during, or after the carrier is combined with the lipid vesicle particles. If the carrier is an emulsion, the polynucleotide may be mixed with either or both of the aqueous phase or hydrophobic phase prior to emulsification. Alternatively, the polynucleotide may be mixed with the carrier after emulsification.

The technique of combining the polynucleotide with the carrier may be used together with encapsulation of the polynucleotide in the lipid vesicle particles as described above, such that polynucleotide is present both within the lipid vesicle particles and in the carrier comprising a continuous phase of a hydrophobic substance.

Generally, the composition may comprise about 0.1 to 5 mg polynucleotide per ml of the composition and about 1 mg to 300 mg lipid vesicle particles per ml of the composition.

In some embodiments, the particle size of the lipid vesicle particles prepared according the methods described herein prior to dehydration is about 100-5000 nm (0.1 to 5 microns). The particle size of the lipid vesicle particles may vary depending upon the ratio of organic:aqueous or aqueous:organic solvent composition in the final formulation prior to dehydration and also on the choice of organic solvent. In some embodiments, the particle size of the lipid vesicle particles prepared according the methods described herein prior to dehydration is about 100-200 nm, about 100-300 nm, about 100-400 nm, about 100-500 nm, about 100-1000 nm, about 100-1500 nm, about 110-1500 nm, about 110-1800 nm, about 120-1500 nm, about 120-2000 nm, about 130-2000 nm, about 130-2500 nm, about 150-3000 nm, about 150-4000 nm, about 200-4000 nm, or about 200-5000 nm.

In some embodiments, the mean particle size of the lipid vesicle particles prepared according the methods described herein prior to dehydration is about 100-1000 nm (0.1 to 1 microns). In some embodiments, the mean particle size of the lipid vesicle particles prepared according the methods described herein prior to dehydration is about 100 nm, about 115 nm, about 120 nm, about 125 nm, about 130 nm, about 135 nm, about 140 nm, about 145 nm, about 150 nm, about 160 nm, about 170 nm, about 180 nm, about 190 nm, about 200 nm, about 300 nm, about 400 nm, about 500 nm, about 600 nm, about 700 nm, about 800 nm, about 900 nm, or about 1000 nm.

In some embodiments, the lipid vesicle particles prepared according the methods described herein after reconstitution in a carrier comprising a continuous phase of a hydrophobic substance form reverse micelles.

In some embodiments, the particle size of the lipid vesicle particles prepared according the methods described herein after reconstitution in a carrier comprising a continuous phase of a hydrophobic substance is about 1-50 nm. In some embodiments, the particle size of the lipid vesicle particles prepared according the methods described herein after reconstitution in a carrier comprising a continuous phase of a hydrophobic substance is about 1-10 nm, 2-8 nm, 4-9 nm, 5-10 nm, 6-12 nm, 7-15 nm, 8-20 nm, 10-30 nm, 15-40 nm, 20-45 nm, or 30-50 nm.

In some embodiments, the mean particle size of the lipid vesicle particles prepared according the methods described herein after reconstitution in a carrier comprising a continuous phase of a hydrophobic substance is about 1-20 nm. In some embodiments, the mean particle size of the lipid vesicle particles prepared according the methods described herein after reconstitution in a carrier comprising a continuous phase of a hydrophobic substance is about 1 nm, about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, or about 20 nm.

There are several techniques, instruments and services that are available to measure the mean particle size of lipid vesicle particles, such as electron microscopy (transmission, TEM, or scanning, SEM), atomic force microscopy (AFM), Fourier-transform infrared spectroscopy (FTIR), X-ray photoelectron spectroscopy (XPS), powder X-ray diffraction (XRD), matrix-assisted laser desorption/ionization time-of-flight mass spectrometry (MALDI-TOF-MS), nuclear magnetic resonance (NMR) and dynamic light scattering (DLS). DLS is a well-established technique for measuring the particle size in the submicron size range, with available technology to measure particle sizes of less than 1 nm (LS Instruments, CH; Malvern Instruments, UK).

Polydispersity index (PDI) is a measure of the size distribution of the lipid vesicle particles. It is known in the art that the term “polydispersity” may be used interchangeably with “dispersity”. The PDI can be calculated by determining the mean particle size of the lipid vesicle particles and the standard deviation from that size. There are techniques and instruments available for measuring the PDI of lipid vesicle particles. For example, DLS is a well-established technique for measuring the particle size and size distribution of particles in the submicron size range, with available technology to measure particle sizes of less than 1 nm (LS Instruments, CH; Malvern Instruments, UK). For a perfectly uniform sample, the PDI would be 0.0.

In some embodiments, PDI of a lipid vesicle particle prepared according the methods described herein prior to dehydration is between about 0.1 to about 0.7. In some embodiments, PDI of a lipid vesicle particle prepared according the methods described herein prior to dehydration is about 0.1 to about 0.4, about 0.2 to about 0.5, about 0.3 to about 0.6, about 0.4 to about 0.7, or about 0.5 to 0.7. In some embodiments, PDI of a lipid vesicle particle described herein is about 0.1, about 0.2, about 0.3, about 0.4, about 0.5, about 0.6, or about 0.7.

In an embodiment, the PDI is measured by any instrument and/or machine suitable for measuring the PDI of lipid vesicle particles. In an embodiment, the PDI size distribution is determined by DLS (Malvern Instruments, UK). In an embodiment, the PDI is measured by DLS using a Malvern Zetasizer series instrument, such as for example the Zetasizer Nano S, Zetasizer APS, Zetasizer pV or Zetasizer AT machines (Malvern Instruments, UK). In an embodiment, the PDI is measured by DLS using a Malvern Zetasizer Nano S machine. Exemplary conditions and system settings are described above in respect of determining mean particle size.

If the composition contains one or more adjuvants and/or additional components, the adjuvant(s) and/or additional component(s) can be incorporated in the composition together with the polynucleotide at the same processing step, or separately, at a different processing step. For instance, the polynucleotide and the adjuvant(s) and/or additional component(s) may both be present in the aqueous solution used to hydrate the lipid bilayer-forming components, such that both the polynucleotide and adjuvant(s) and/or additional component(s) become encapsulated in the lipid vesicle particles. Alternatively, the polynucleotide may be encapsulated in the lipid vesicle particles, and the adjuvant(s) and/or additional component(s) mixed with the carrier comprising a continuous phase of a hydrophobic substance. It will be appreciated that many such combinations are possible.

In some embodiments, the polynucleotide and the adjuvant(s) and/or additional component(s) may be in the form of a complex, in which they are in intimate contact at least prior to incorporation into the composition. Complexing may but need not necessarily involve a chemical linkage, such as covalent bonding.

The compositions as described herein may be formulated in a form that is suitable for any administration routes, such as oral, nasal, aerosol, rectal or parenteral administration. Parenteral administration includes intravenous, intraperitoneal, intradermal, subcutaneous, intramuscular, transepithelial, intrapulmonary, intrathecal, and topical modes of administration. The compositions may be formulated for systemic or localized distribution in the body of the subject. The preferred routes are intramuscular, subcutaneous and intradermal to achieve a depot effect. In practice, a depot effect is achieved when the therapeutic agent remains at the site of injection for more than about one hour.

The injection site may be anywhere close to, or directly into a lymph node, for example. Alternatively, the injection site may be directly into a spleen, a tumor or other diseased tissue. The volume that may be injected is within the professional judgment of the clinician. The volume depends on the injecting device used and the site of injection. When the injection is intramuscularly or subcutaneous, the injection volume may be about 1 mL. When needleless injection is used, the volume may be as low as 0.01 mL. The volume may be increased by injecting multiple sites. Suitable injection volumes may be about 0.01 mL, about 0.02 mL, about 0.05 mL, about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.5 mL, about 2 mL, about 0.01-0.05 mL, about 0.05-0.1 mL, about 0.1-0.2 mL, about 0.2-0.4 mL, about 0.1-0.5 mL, about 0.4-0.8 mL, about 0.5-1 mL, about 0.8-1.2 mL, or about 1-1.5 mL.

Upon administration, formulations of the present disclosure may create stable depot at the site of delivery that protects active ingredient (e.g., a polynucleotide) from degradation by nucleases or by renal or hepatic clearance for an extended period of time (e.g., up to one week or two weeks). In some embodiments, formulations of the present disclosure may also provide controlled and prolonged exposure of active ingredients (e.g., a polynucleotide) to the cells at the delivery site allowing a direct delivery of treatment into target and limits drug distribution specifically to its target site, thus avoiding systemic effects.

### Kits and Reagents

The present disclosure may be optionally provided to a user as a kit. For example, a kit of the disclosure contains one or more of the compositions of the disclosure. The kit can further comprise one or more additional reagents, packaging material, containers for holding the components of the kit, and an instruction set or user manual detailing preferred methods of using the kit components for a desired purpose.

### Methods of the Invention

The invention finds application in any instance in which it is desired to deliver a negatively charged molecule (e.g., polynucleotide) to a target cell or a subject.

In one aspect, provided herein is a method for delivering a negatively charged molecule to a target cell, comprising administering a composition of the present disclosure to the target cell. In some embodiments, the target cell is an antigen-presenting cell (APC).

In another aspect, provided herein is a method for delivering a negatively charged molecule to a subject, comprising administering the composition of the present disclosure to the subject.

The compositions of the present disclosure may have applications in the treatment or prevention of a disease. Representative applications of the disclosure include cancer treatment and prevention, gene therapy, adjuvant therapy, infectious disease treatment and prevention, allergy treatment and prevention, autoimmune disease treatment and prevention, neuron-degenerative disease treatment, and arteriosclerosis treatment.

Prevention or treatment of disease includes obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, conferring protective immunity against a disease-causing agent and amelioration or palliation of the disease state. Prevention or treatment can also mean prolonging survival of a patient beyond that expected in the absence of treatment and can also mean inhibiting the progression of disease temporarily, although more preferably, it involves preventing the occurrence of disease such as by preventing infection in a subject.

In another embodiment, the antigen encoded by a polynucleotide in the composition of the disclosure may be a cancer or tumor-associated protein, such as for example, a membrane surface-bound cancer antigen which is capable of being recognized by an antibody.

Cancers that may be treated and/or prevented by the use or administration of a composition of the disclosure include, without limitation, carcinoma, adenocarcinoma, lymphoma, leukemia, sarcoma, blastoma, myeloma, and germ cell tumors. In one embodiment, the cancer may be caused by a pathogen, such as a virus. Viruses linked to the development of cancer are known to the skilled person and include, but are not limited to, human papillomaviruses (HPV), John Cunningham virus (JCV), Human herpes virus 8, Epstein Barr Virus (EBV), Merkel cell polyomavirus, Hepatitis C Virus and Human T cell leukemia virus-1. A composition of the disclosure may be used for either the treatment or prophylaxis of cancer, for example, in the reduction of the severity of cancer or the prevention of cancer recurrences. Cancers that may benefit from the compositions of the disclosure include any malignant cell that expresses one or more tumor specific antigens.

In another embodiment, the antigen may be a toxin or an allergen that is capable of being neutralized by an antibody.

In another embodiment, the antigen may be an antigen associated with a disease where it is desirable to sequester the antigen in circulation, such as for example an amyloid protein (e.g., Alzheimer's disease). Thus, a composition of the disclosure may be suitable for use in the treatment and/or prevention of a neurodegenerative disease in a subject in need thereof, wherein the neurodegenerative disease is associated with the expression of an antigen. The subject may have a neurodegenerative disease or may be at risk of developing a neurodegenerative disease. Neurodegenerative diseases that may be treated and/or prevented by the use or administration of a composition of the disclosure include, without limitation, Alzheimer's disease, Parkinson's disease, Huntington's disease, and amyotrophic lateral sclerosis (ALS). For example, Alzheimer's disease is characterized by the association of B-amyloid plaques and/or tau proteins in the brains of patients with Alzheimer's disease (see, for example, Goedert and Spillantini, Science, 314: 777-781, 2006). Herpes simplex virus type 1 has also been proposed to play a causative role in people carrying the susceptible versions of the apoE gene (Itzhaki and Wozniak, J Alzheimers Dis 13: 393-405, 2008).

In a further embodiment, the composition may comprise a mixture of B cell epitopes as antigens for inducing a humoral immune response. The B cell epitopes may be linked to form a single polypeptide.

In another embodiment, the antigen may be any peptide or polypeptide that is capable of inducing a specific humoral immune response to a specific conformation on targeted tumor cells.

In some embodiments, the composition of the present disclosure may be used to induce humoral and/or cellular immune response in a subject. Accordingly, compositions as described herein may be useful for treating or preventing diseases and/or disorders ameliorated by humoral immune responses (e.g., involving B-cells and antibody production). The compositions may find application in any instance in which it is desired to administer an antigen to a subject to induce a humoral immune response or antibody production.

A humoral immune response, as opposed to cell-mediated immunity, is mediated by secreted antibodies which are produced in the cells of the B lymphocyte lineage (B cells). Such secreted antibodies bind to antigens, such as for example those on the surfaces of foreign substances and/or pathogens (e.g., viruses, bacteria, etc.) and flag them for destruction.

Antibodies are the antigen-specific glycoprotein products of a subset of white blood cells called B lymphocytes (B cells). Engagement of antigen with antibody expressed on the surface of B cells can induce an antibody response comprising stimulation of B cells to become activated, to undergo mitosis and to terminally differentiate into plasma cells, which are specialized for synthesis and secretion of antigen-specific antibody.

B cells are the sole producers of antibodies during an immune response and are thus a key element to effective humoral immunity. In addition to producing large amounts of antibodies, B cells also act as antigen-presenting cells and can present antigen to T cells, such as T helper CD4 or cytotoxic CD8, thus propagating the immune response. B cells, as well as T cells, are part of the adaptive immune response which is essential for vaccine efficacy. During an active immune response, induced either by vaccination or natural infection, antigen-specific B cells are activated and clonally expand. During expansion, B cells evolve to have higher affinity for the epitope. Proliferation of B cells can be induced indirectly by activated T-helper cells, and also directly through stimulation of receptors, such as the toll-like receptors (TLRs).

Antigen presenting cells, such as dendritic cells, macrophages and B cells, are drawn to vaccination sites and can interact with antigens and adjuvants contained in the vaccine. The adjuvant stimulates the cells to become activated and the antigen provides the blueprint for the target. Different types of adjuvants provide different stimulation signals to cells. For example, Poly I.C (a TLR3 agonist) can activate dendritic cells, but not B cells. Adjuvants such as Pam3Cys, Pam2Cys and FSL-1 are especially adept at activating and initiating proliferation of B cells, which is expected to facilitate the production of an antibody response (Moyle et al., Curr Med Chem, 2008; So., J Immunol, 2012, which are incorporated hereby by reference in their entireties).

One method of evaluating an antibody response is to measure the titers of antibodies reactive with a particular antigen. This may be performed using a variety of methods known in the art such as enzyme-linked immunosorbent assay (ELISA) of antibody-containing substances obtained from animals. For example, the titers of serum antibodies which bind to a particular antigen may be determined in a subject both before and after exposure to the antigen. A statistically significant increase in the titer of antigen-specific antibodies following exposure to the antigen would indicate the subject had mounted an antibody response to the antigen.

Other assays that may be used to detect the presence of an antigen-specific antibody include, without limitation, immunological assays (e.g., radioimmunoassay (RIA)), immunoprecipitation assays, and protein blot (e.g., Western blot) assays; and neutralization assays (e.g., neutralization of viral infectivity in an in vitro or in vivo assay).

The compositions of the present disclosure, by stimulating strong antibody responses, may be capable of protecting a subject from a disease, disorder or ailment associated with an antigen capable of inducing a humoral immune response.

Without limitation, this includes for example, infectious diseases, cancers involving a membrane surface-bound cancer antigen which is recognized by an antibody, diseases where it is desirable to sequester antigen in circulation, like amyloid protein (e.g., Alzheimer's disease); neutralizing toxins with an antibody; neutralizing viruses or bacteria with an antibody; or neutralizing allergens (e.g., pollen) for the treatment of allergies.

A humoral immune response is the main mechanism for effective infectious disease vaccines. However, a humoral immune response can also be useful for combating cancer. Unlike a cancer vaccine designed to produce a cytotoxic CD8 T cell response that can recognize and destroy cancer cells, B cell mediated responses may target cancer cells through other mechanisms which may in some instances cooperate with a cytotoxic CD8 T cell for maximum benefit. Examples of mechanisms of B cell mediated (e.g., humoral immune response mediated) anti-tumor responses include, without limitation: 1) Antibodies produced by B cells that bind to surface antigens found on tumor cells or other cells that influence tumorigenesis. Such antibodies can, for example. induce killing of target cells through antibody-dependent cell-mediated cytotoxicity (ADCC) or complement fixation, potentially resulting in the release of additional antigens that can be recognized by the immune system; 2) Antibodies that bind to receptors on tumor cells to block their stimulation and in effect neutralize their effects; 3) Antibodies that bind to factors released by or associated with tumor or tumor-associated cells to modulate a signaling or cellular pathway that supports cancer; and 4) Antibodies that bind to intracellular targets and mediate anti-tumor activity through a currently unknown mechanism.

Several methods can be used to demonstrate the induction of humoral immunity following vaccination. These can be broadly classified into detection of: i) specific antigen presenting cells; ii) specific effector cells and their functions; and iii) release of soluble mediators such as cytokines.

In various embodiments, the composition may be administered via oral, nasal, rectal or parenteral administration. Parenteral administration includes intravenous, intraperitoneal, intradermal, subcutaneous, intramuscular, transepithelial, intrapulmonary, intrathecal, and topical modes of administration. In some embodiments, the composition is administered via intramuscular, subcutaneous or intradermal injection.

The amount of composition used in a single treatment may vary depending on factor such as the nature of negatively charged molecule to be delivered, the type of formulation, and the size of the subject. One skilled in the art will be able to determine, without undue experimentation, the effective amount of composition to use in a particular application.

The skilled artisan can determine suitable treatment regimes, routes of administration, dosages, etc., for any particular application in order to achieve the desired result. Factors that may be taken into account include, e.g., the nature of a polypeptide to be expressed; the disease state to be prevented or treated; the age, physical condition, body weight, sex and diet of the subject; and other clinical factors.

The subject to be treated may be any vertebrate, preferably a mammal, more preferably a human.

### EXEMPLARY EMBODIMENTS

1. A composition, comprising:
   a) one or more lipids,
   b) a negatively charged molecule,
   c) a carrier comprising a continuous phase of a hydrophobic substance, and
   d) an ionizable aminoglycoside.
2. The composition of embodiment 1, wherein the one or more lipids comprise one or more of a phospholipid, cholesterol or a cholesterol derivative, or a combination thereof.
3. The composition of embodiment 2, wherein the phospholipid is one or more of phosphoglycerol, phosphoethanolamine, phosphoserine, phosphocholine, phosphoinositol, phosphatidylcholine or lecithin.
4. The composition of embodiment 3, wherein the phospholipid comprises dioleoyl phosphatidylcholine (DOPC), 1,2-dipalmitoyl-sn-glycero-3-phosphocholine (DPPC), dioleoyl phosphatidylethanolamine (DOPE), 1,2-dipalmitoyl-sn-glycero-3-succinate (DGS), or a combination thereof.
5. The composition of any one of embodiments 2-4, wherein the one or more lipids comprise DOPC and cholesterol.
6. The composition of any one of embodiments 1-5, wherein the ionizable aminoglycoside is one or more of chitosan, cationic alginate, cationic gelatin, cationic dextran, diethylaminoethyl (DEAE)-dextran hydrochloride, aminated cellulose, aminated sucrose, aminated trehalose, N-acetyl-D-glucosamine, D-(+)-glucosamine hydrochloride, trehalose-6,6-dibehenate (TDB) with Dimethyldioctadecylammonium (DDA), heptakis(6-deoxy-6-amino)-β-cyclodextrin heptahydrochloride, and glycyrrhizic acid ammonium salt, or derivatives thereof.
7. The composition of embodiment 6, wherein the ionizable aminoglycoside is chitosan.
8. The composition of embodiment 7, wherein the chitosan has a molecular weight of about 60 kDa to 150 kDa.
9. The composition of embodiment 7 or 8, wherein the chitosan has a molecular weight of about 100 kDa to 120 kDa.
10. The composition of any one of embodiments 7-9, wherein the chitosan has a molecular weight of about 100 kDa.
11. The composition of any one of embodiments 7-10, wherein the chitosan has a degree of deacetylation (DD) of about 15-95%.
12. The composition of embodiment 11, wherein the chitosan has a degree of deacetylation (DD) of about 25%.
13. The composition of any one of embodiments 7-12, wherein the chitosan is added in a concentration of about 0.5 mg/mL to about 3 mg/mL.
14. The composition of any one of embodiments 7-13, wherein the chitosan is added in a concentration of about 1 mg/mL to about 2 mg/mL.
15. The composition of any one of embodiments 1-14, further comprising an adjuvant.
16. The composition of embodiments 1 and 6-14, comprising:
   a) one or more positively charged lipids,
   b) a negatively charged molecule,
   c) a carrier comprising a continuous phase of a hydrophobic substance,
   d) an ionizable aminoglycoside, and
   e) optionally, an adjuvant.
17. The composition according to embodiment 16, wherein the one or more positively charged lipids comprise 1,2-dioleoyl-3-trimethylammonium-propane (DOTAP), 30-[N—(N′,N′-dimethylaminoethane)-carbamoyl]cholesterol (DC-cholesterol), 1,2-distearoyl-3-dimethylammonium-propane (DAP), N-(4-carboxybenzyl)-N,N-dimethyl-2,3-bis(oleoyloxy)propan-1-aminium (DOBAQ), N-palmitoyl homocysteine (PHC), DC-cholesterol, or a combination thereof.
18. The composition of any one of embodiments 1-17, wherein the negatively charged molecule is a polynucleotide.
19. The composition of any one of embodiments 1-18, wherein the negatively charged molecule is a ribonucleic acid (RNA), or RNA derivative.
20. The composition of any one of embodiments 1-18, wherein the negatively charged molecule is a deoxyribonucleic acid (DNA), or DNA derivative.
21. The composition of any one of embodiments 18-20, wherein the polynucleotide comprises or encodes a messenger RNA (mRNA), an antisense RNA, an interfering RNA, a catalytic RNA, or a ribozyme.
22. The composition of embodiment 21, wherein the polynucleotide comprises an mRNA.
23. The composition of any one of embodiments 18-22, wherein the polynucleotide encodes a polypeptide.
24. The composition of embodiment 23, wherein the polypeptide is an antigen, an antibody or antibody fragment, an enzyme, a cytokine, a therapeutic protein, a chemokine, a regulatory protein, a structural protein, a chimeric protein, a nuclear protein, a transcription factor, a viral protein, a TLR protein, an interferon regulatory factor, an angiostatic or angiogenic protein, an apoptotic protein, an Fc gamma receptor, a hematopoietic protein, a tumor suppressor, a cytokine receptor, or a chemokine receptor.
25. The composition of embodiment 24, wherein the antigen is derived from a virus, bacterium or protozoan, a membrane surface-bound cancer antigen, a toxin, or an allergen.
26. The composition of any one of embodiments 1-25, wherein the concentration ratio of the lipids and the negatively charged molecule is between about 33000:1 to about 3300:1.
27. The composition of embodiment 26, wherein the concentration ratio of the lipids and the negatively charged molecule is between about 26400:1 to about 6600:1.
28. The composition of embodiment 27, wherein the concentration ratio of the lipids and the negatively charged molecule is about 13200:1.
29. The composition of any one of embodiments 1-28, wherein the carrier comprises an oil or a water-in-oil emulsion.
30. The composition of embodiment 29, wherein the oil comprises a natural oil or a synthetic oil.
31. The composition of embodiment 30, wherein the oil comprises a vegetable oil, mineral oil, a nut oil, soybean oil, peanut oil, or combinations thereof.
32. The composition of embodiment 31, wherein the carrier comprises a mannide oleate in mineral oil solution.
33. The composition of embodiment 32, wherein the carrier comprises Montanide® ISA 51.
34. The composition of embodiment 31, wherein the carrier comprises MS80 oil (mixture of mineral oil and Span 80).
35. The composition of any one of embodiments 15-34, wherein the adjuvant is a polymer, a protein, a polysaccharide, or a combination thereof.
36. The composition of any one of embodiments 1-35, further comprising a buffer and/or surfactant.
37. The composition of any one of embodiments 1-36, wherein the composition is an injectable composition.
38. A method for delivering a negatively charged molecule to a target cell, comprising administering the composition of any one of embodiments 1-37 to said target cell.
39. The method of embodiment 38, wherein the target cell is an antigen-presenting cell (APC).
40. A method for delivering a negatively charged molecule to a subject, comprising administering the composition of any one of embodiments 1-37 to said subject.
41. A method for treating or preventing cancer, an infectious disease or an disease and/or disorder ameliorated by humoral and/or cellular immune response in a subject in need thereof, said method comprising administering to the subject an effective amount of the composition of any one of embodiments 1-37.
42. The method of any one of embodiments 38-40, wherein the negatively charged molecule is a polynucleotide.
43. The method of embodiment 42, wherein the polynucleotide comprises an mRNA.
44. The method of embodiment 42 or 43, wherein the polynucleotide encodes a polypeptide.
45. The method of embodiment 44, wherein the polypeptide is an antigen, an antibody or antibody fragment, an enzyme, a cytokine, a therapeutic protein, a chemokine, a regulatory protein, a structural protein, a chimeric protein, a nuclear protein, a transcription factor, a viral protein, a TLR protein, an interferon regulatory factor, an angiostatic or angiogenic protein, an apoptotic protein, an Fc gamma receptor, a hematopoietic protein, a tumor suppressor, a cytokine receptor, or a chemokine receptor.
46. The method of embodiment 45, wherein the antigen is derived from a virus, bacterium or protozoan, a membrane surface-bound cancer antigen or a toxin.
47. The method of any one of embodiment 40-46, wherein the composition is administered via subcutaneous, intramuscular or intradermal injection.
48. A method for preparing a composition of any one of embodiments 1-37.
49. A method for preparing a composition comprising one or more lipids, a negatively charged molecule, a carrier comprising a continuous phase of a hydrophobic substance, and an ionizable aminoglycoside, comprising:
   a) dissolving the one or more lipids in one or more organic solvents and optionally an aqueous solvent to create a lipid solution,
   b) adding the negatively charged molecule to the lipid solution formed in step a) and mixing;
   c) adding the ionizable aminoglycoside to the mixture formed in step b) and mixing;
   d) optionally, adding additional amount of the organic solvent(s) or aqueous solvent to the mixture formed in step c) thereby the overall Wt/Wt or V/V percentage ratio of organic:aqueous solvent or aqueous:organic solvent in the mixture is between 20-50%;
   e) drying the mixture formed in step c) or d) to generate a dried preparation; and
   f) dissolving the dried preparation in the carrier comprising a continuous phase of a hydrophobic substance, thereby generating said composition.
50. The method of embodiment 49, wherein in step a) the one or more organic solvents is present in an amount sufficient to prevent the one or more lipids from forming lipid vesicle particles in the lipid solution.
51. The method of embodiment 49 or 50, wherein the one or more organic solvents comprises tert-butanol, ethanol, methanol, chloroform, or a mixture thereof.
52. The method of embodiment 49 or 50, wherein the one or more organic solvent comprises tert-butanol, tert-butanol-ethanol mixture or tert-butanol-chloroform mixture.
53. The method of any one of embodiments 49-52, wherein the lipid solution comprises about 30% tert-butanol.
54. The method of any one of embodiments 49-53, wherein the aqueous solvent is water or a buffer solution.
55. The method of any one of embodiments 49-54, wherein the negatively charged molecule is present in an aqueous solution.
56. The method of any one of embodiments 49-55, wherein the ionizable aminoglycoside is present in an aqueous solution.
57. The method of any one of embodiments 49-56, wherein drying is performed by freeze-drying, spray freeze-drying, spray drying, or rotary evaporation.
58. The method of any one of embodiments 49-57, wherein the one or more lipids comprise one or more of a phospholipid, cholesterol or a cholesterol derivative, or a combination thereof.
59. The method of embodiment 58, wherein the phospholipid is one or more of phosphoglycerol, phosphoethanolamine, phosphoserine, phosphocholine, phosphoinositol, phosphatidylcholine or lecithin.
60. The method of embodiment 59, wherein the phospholipid comprises dioleoyl phosphatidylcholine (DOPC), 1,2-dipalmitoyl-sn-glycero-3-phosphocholine (DPPC), dioleoyl phosphatidylethanolamine (DOPE), 1,2-dipalmitoyl-sn-glycero-3-succinate (DGS), or a combination thereof.
61. The method of any one of embodiments 58-60, wherein the one or more lipids comprise DOPC and cholesterol.
62. The method of any one of embodiments 49-61, wherein the ionizable aminoglycoside is one or more of chitosan, cationic alginate, cationic gelatin, cationic dextran, diethylaminoethyl (DEAE)-dextran hydrochloride, aminated cellulose, aminated sucrose, aminated trehalose, N-acetyl-D-glucosamine, D-(+)-glucosamine hydrochloride, trehalose-6,6-dibehenate (TDB) with Dimethyldioctadecylammonium bromide (DDA), heptakis(6-deoxy-6-amino)-p-cyclodextrin heptahydrochloride, and glycyrrhizic acid ammonium salt, or derivatives thereof.
63. The method of embodiment 62, wherein the ionizable aminoglycoside is chitosan.
64. The method of embodiment 63, wherein the chitosan has a molecular weight of about 60 kDa to 150 kDa.
65. The method of embodiment 62 or 63, wherein the chitosan has a molecular weight of about 100 kDa to 120 kDa.
66. The method of any one of embodiments 63-65, wherein the chitosan has a molecular weight of about 100 kDa.
67. The method of any one of embodiments 63-66, wherein the chitosan has a degree of deacetylation (DD) of about 15-95%.
68. The method of embodiment 67, wherein the chitosan has a degree of deacetylation (DD) of about 25%.
69. The method of any one of embodiments 63-68, wherein the chitosan is added in a concentration of about 0.5 mg/mL to about 3 mg/mL.
70. The method of any one of embodiments 63-69, wherein the chitosan is added in a concentration of about 1 mg/mL to about 2 mg/mL.
71. The method of any one of embodiments 49-70, wherein the negatively charged molecule is a polynucleotide.
72. The method of any one of embodiments 49-71, wherein the negatively charged molecule is a ribonucleic acid (RNA), or RNA derivative.
73. The method of any one of embodiments 49-72, wherein the negatively charged molecule is a deoxyribonucleic acid (DNA), or DNA derivative.
74. The method of any one of embodiments 71-73, wherein the polynucleotide comprises or encodes a messenger RNA (mRNA), an antisense RNA, an interfering RNA, a catalytic RNA, or a ribozyme.
75. The method of embodiment 74, wherein the polynucleotide comprises an mRNA.
76. The method of any one of embodiments 72-75, wherein the polynucleotide encodes a polypeptide.
77. The method of embodiment 76, wherein the polypeptide is an antigen, an antibody or antibody fragment, an enzyme, a cytokine, a therapeutic protein, a chemokine, a regulatory protein, a structural protein, a chimeric protein, a nuclear protein, a transcription factor, a viral protein, a TLR protein, an interferon regulatory factor, an angiostatic or angiogenic protein, an apoptotic protein, an Fc gamma receptor, a hematopoietic protein, a tumor suppressor, a cytokine receptor, or a chemokine receptor.
78. The method of embodiment 77, wherein the antigen is derived from a virus, bacterium or protozoan, a membrane surface-bound cancer antigen, a toxin, or an allergen.
79. The method of any one of embodiments 49-78, wherein the concentration ratio of the lipids and the negatively charged molecule is between about 33000:1 to about 3300:1.
80. The method of embodiment 79, wherein the concentration ratio of the lipids and the negatively charged molecule is between about 26400:1 to about 6600:1.
81. The method of embodiment 80, wherein the concentration ratio of the lipids and the negatively charged molecule is about 13200:1.
82. The method of any one of embodiments 49-81, wherein the carrier comprises an oil or a water-in-oil emulsion.
83. The method of embodiment 82, wherein the oil comprises a natural oil or a synthetic oil.
84. The method of embodiment 83, wherein the oil comprises a vegetable oil, mineral oil, a nut oil, soybean oil, peanut oil, or combinations thereof.
85. The method of embodiment 84, wherein the carrier comprises a mannide oleate in mineral oil solution.
86. The method of embodiment 85, wherein the carrier comprises Montanide® ISA 51.
87. The method of embodiment 84, wherein the carrier comprises MS80 oil (mixture of mineral oil and Span 80).
88. The method of any one of embodiments 49-87, wherein the composition further comprises an adjuvant.
89. The method of embodiment 88, wherein the adjuvant is a polymer, a protein, a polysaccharide, or a combination thereof.
90. The method of any one of embodiments 49-89, wherein the composition further comprises a buffer and/or surfactant.
91. The method of any one of embodiments 49-90, wherein the composition is an injectable composition.
92. A kit comprising a composition of any one of embodiments 1-37, and instructions for using said composition to deliver a negatively charged molecule to a subject.
93. The method of any one of embodiments 40-47 or the kit of embodiment 92, wherein said subject is a mammal.
94. The method of any one of embodiments 40-47 or the kit of embodiment 92, wherein said subject is a human.

### EXAMPLES

The following examples are provided to further describe some of the embodiments disclosed herein. The examples are intended to illustrate, not to limit, the disclosed embodiments.

### Example 1. Addition of Different Lipids/Polymers/Transfection Agents in Nucleic Acid Formulation

Seven test formulations were prepared with the addition of different lipids, polymers, or transfection agents to the standard formulation (see Formulation Method A). The methods and materials used for preparing each of these formulations are detailed below.

Formulation Method A (Standard)

250 μL of 0.2 M sodium acetate, pH 7 was added to a sterile baked 2-mL vial. Next, 50 μL eGFP mRNA was added and the solution was gently swirled by hand to mix. 500 μL of 132 mg/mL DOPC/Chol lipid vesicle particles (Size 85.05 nm, polydispersity index (PDI) 0.047, pH 6.36) prepared in diethylpyrocarbonate (DEPC) treated water was then added to this eGFP mRNA solution. The solution was gently swirled/shook by hand to mix and filled to 1.0 mL by adding 200 μL of DEPC treated water. The vial was then partially stoppered and freeze-dried. The freeze-dried cake was then reconstituted with 0.45 mL of Montanide® ISA 51 oil diluent to obtain final concentrations of DOPC/Chol 132 mg/mL, eGFPmRNA 0.1 mg/mL and sodium acetate 0.1 M. A 50 μL of this formulation was injected subcutaneously (SC) in mice.

Formulation Method A with Transfection Agent Protamine 0.5%

2.122 mg of Protamine using BL-5 Sartorius Microbalance was weighed in a sterile baked 2-mL vial. 380.38 μL of DEPC treated water was added. The vial was vortexed for 2 minutes and incubated at 37° C. with shaking at 200 revolutions per minute (RPM) for 30 mins. Next, 212.5 μL of 0.2 M sodium acetate, pH 7 was added to the prepared protamine solution and mixed well. 212.5 μL of 132 mg/mL DOPC/Chol lipid vesicle particles in DEPC treated water (Size 85.05 nm, PDI 0.047, pH 6.36) was added. The solution was gently swirled/shook by hand to mix. The pH was adjusted to 7 and 42.5 μL eGFP mRNA was added. The solution was gently swirled by hand to mix and the vial was freeze-dried. The freeze-dried cake was then reconstituted with 0.397 mL of Montanide® ISA 51 oil diluent to obtain final concentrations of DOPC/Chol 66 mg/mL, eGFP mRNA 0.1 mg/mL, Protamine 0.5% and sodium acetate 0.1 M. A 50 μL of this formulation was injected SC in mice.

Formulation Method A with Transfection Agent Mannose 2%

212.5 μL of 0.2 M sodium acetate, pH 7 was added to a 2-mL baked vial. Next, 212.5 μL of 132 mg/mL DOPC/Chol lipid vesicle particles in DEPC treated water (Size 85.05 nm, PDI 0.047, pH 6.36) was added. The solution was gently swirled/shook by hand to mix. 85 μL of 10% mannose stock (dissolved 119.1 mg of Mannose in 1.19 mL DEPC treated water and vortexed for 1 minute to obtain clear solution) was added and the solution was gently swirled by hand to mix. pH was adjusted to 6.5 and then 297.5 μL of DEPC treated water was added. Finally, 42.5 μL eGFP-mRNA was added, the solution was gently swirled by hand and the vial was freeze-dried. The freeze-dried cake was then reconstituted with 0.397 mL of Montanide® ISA 51 oil diluent to obtain final concentrations of DOPC/Chol 66 mg/mL, eGFP mRNA 0.1 mg/mL, Mannose 2% and sodium acetate 0.1 M. A 50 μL of this formulation was injected SC in mice.

Formulation Method A with Transfection Agent Chloroquine 1%

212.5 μL of 0.2M Sodium Acetate, pH 7 was added to a 2-mL baked vial. Next, 212.5 μL of 132 mg/mL DOPC/Chol lipid vesicle particles in DEPC treated water (Size 85.05 nm, PDI 0.047, pH 6.36) was added and the solution was gently swirled/shook by hand to mix. 42.5 μL of 10% chloroquine stock (dissolved 138.1 mg of chloroquine in 1.381 mL 10% acetic acid and vortexed for 2 minutes to obtain clear solution) was added. The solution was gently swirled by hand to mix. pH was adjusted from 4.14 to 7.43 using 160 μL 0.5M NaOH and then 180 μL of DEPC treated water was added. Finally, 42.5 μL eGFP mRNA was added, the solution was gently swirled by hand and the vial was freeze-dried. The freeze-dried cake was then reconstituted with 0.397 mL of Montanide® ISA 51 oil diluent to obtain final concentrations of DOPC/Chol 66 mg/mL, eGFP-mRNA 0.1 mg/mL, Chloroquine 10% and sodium acetate 0.1 M. A 50 μL of this formulation was injected SC in mice.

Formulation Method A with Chitosan Polymer (60-120 kDa/15-25% DD; 1 mg/mL)

212.5 μL of 0.2 M sodium acetate, pH 7 was added to a sterile baked 2-mL vial. Next, 42.5 μL eGFP mRNA was added and the solution was gently swirled by hand to mix. 212.5 μL of 132 mg/mL DOPC/Chol lipid vesicle particles (Size 85.05 nm, PDI 0.047, pH 6.36) prepared in DEPC treated water was added to this eGFP-mRNA solution. The solution was gently swirled/shook by hand to mix. 42.5 μL of chitosan stock (60-120 kDa/15-25% DD, 10 mg/mL in 0.25% Acetic Acid) was added and the solution was filled to 0.85 mL by adding 340 μL of DEPC treated water. pH was measured as 6.1. The vial was then partially stoppered and freeze-dried. The freeze-dried cake was then reconstituted with 0.397 mL of Montanide® ISA 51 oil diluent to obtain final concentrations of DOPC/Chol 66 mg/mL, eGFP mRNA 0.1 mg/mL, chitosan 1 mg/mL and sodium acetate 0.1 M. A 50 μL of this formulation was injected SC in mice.

Formulation Method A with Cationic Lipid DOTAP

212.5 μL of 0.2 M sodium acetate, pH 7 was added to a sterile baked 2-mL vial. Next, 42.5 μL eGFP-mRNA was added and the solution was gently swirled by hand to mix. 425 μL of 66 mg/mL DOTAP lipid vesicle particles (Size 87.48 nm, PDI 0.059, pH 5.12) prepared in DEPC treated water was added to this eGFP mRNA solution. The solution was gently swirled/shook by hand to mix and filled to 0.85 mL by adding 170 μL of DEPC treated water. The final pH of the formulation was pH 7.1. The vial was then partially stoppered and freeze-dried. The freeze-dried cake was then reconstituted with 0.397 mL of Montanide® ISA 51 oil diluent to obtain final concentrations of DOTAP 132 mg/mL, eGFPmRNA 0.1 mg/mL and sodium acetate 0.1 M. A 50 μL of this formulation was injected SC in mice.

Formulation Method A with Cationic Lipid DOTAP & DOPC/Chol Mixture

212.5 μL of 0.2 M sodium acetate, pH 7 was added to a sterile baked 2-mL vial. Next, 42.5 μL eGFP mRNA was added and the solution was gently swirled by hand to mix. 106.25 μL of 132 mg/mL DOPC/Chol lipid vesicle particles prepared in DEPC treated water (Size 85.05 nm, PDI 0.047, pH 6.36) was added to this eGFP mRNA solution. The solution was gently swirled/shook by hand to mix. Next, 276.25 μL of DEPC treated water was added followed by the addition of 212.5 μL of 66 mg/mL DOTAP nanoparticles prepared in DEPC treated water (Size 87.48 nm, PDI 0.059, pH 5.12). The solution was gently swirled/shook by hand to mix. The final pH of the formulation was pH 6.90. The vial was then partially stoppered and freeze-dried. The freeze-dried cake was then reconstituted with 0.397 mL of Montanide® ISA 51 oil diluent to obtain final concentrations of DOTAP 33 mg/mL, DOPC/Chol 33 mg/mL, eGFP mRNA 0.1 mg/mL and sodium acetate 0.1 M. A 50 μL of this formulation was injected SC in mice.

Formulation Method A with Cationic Lipid DC-Cholesterol

212.5 μL of 0.2 M sodium acetate, pH 7 was added to a sterile baked 2-mL vial. Next, 42.5 μL eGFP mRNA was added and gently swirled by hand to mix. 425 μL of 66 mg/mL DC-Cholesterol lipid vesicle particles prepared in DEPC treated water (Size 137.3 nm, PDI 0.038, pH 4.08) was added to this eGFP mRNA solution. The solution was gently swirled/shook by hand to mix. Next, 170 μL of DEPC treated water was added. The solution was gently swirled/shook by hand to mix. The final pH of the formulation was pH 6.2. The vial was then partially stoppered and freeze-dried. The freeze-dried cake was then reconstituted with 0.397 mL of Montanide® ISA 51 oil diluent to obtain final concentrations of DC-Cholesterol 66 mg/mL, eGFP mRNA 0.1 mg/mL and sodium acetate 0.1 M. A 50 μL of this formulation was injected SC in mice.

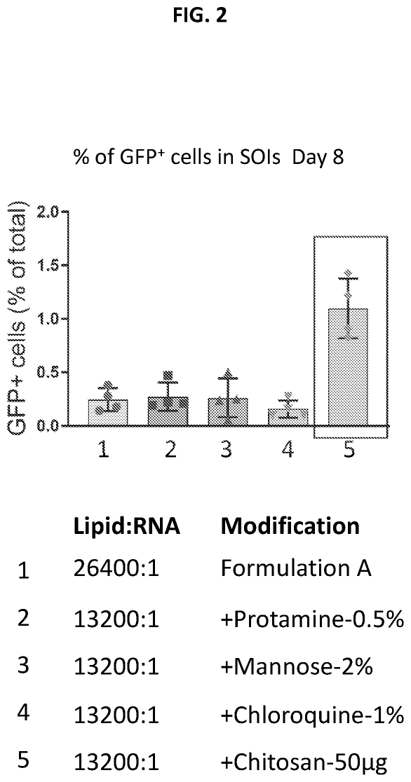

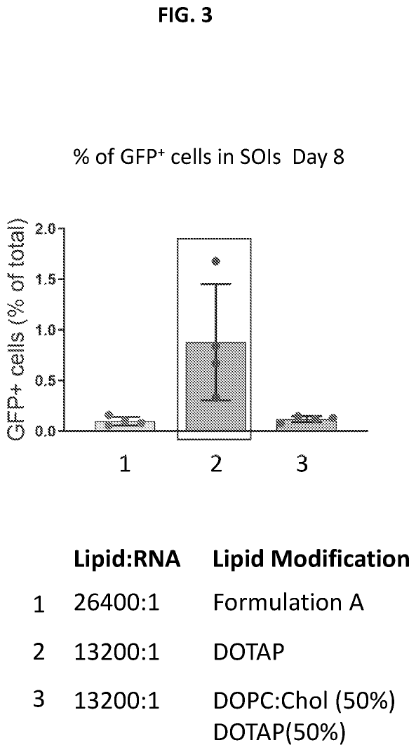

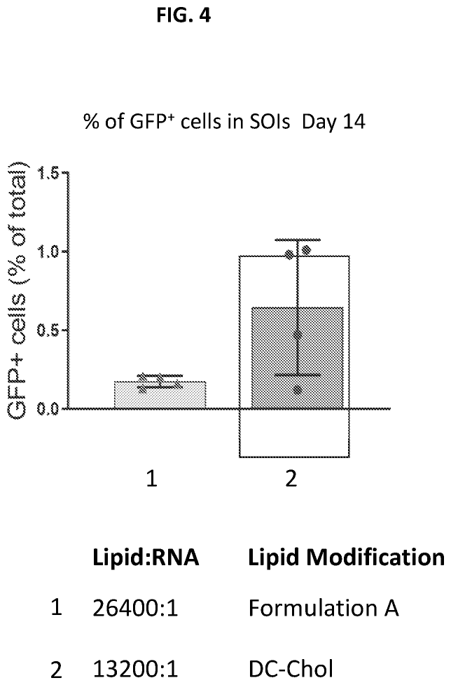

Mice (Female, CD57BL/6, 6-8 weeks, 15-25 g n=4 per group) were treated with 50 μL formulations described above on study day (SD) 0. Formulations were administered subcutaneously in the right flank. Mice were terminated on SD8 or 14 when tissues from the site of injection were collected and processed by crushing in PBS buffer, centrifuging and resuspending in PBS buffer. Intracellular eGFP expression was quantified using a BD FACSCelesta flow cytometer and FlowJo software. Up to 300,000 events was acquired for each sample in the FITC channel using the 518/528 filter. The percent of GFP positive cells in the site of injection (SOI) was determined on SD8 (FIGS. 2 and 3) and Day 14 (FIG. 4) after injection. It was shown that addition of chitosan polymer (FIG. 2) or using cationic lipids DOTAP (FIG. 3), DC-Cholesterol (FIG. 4) in the formulation strongly enhance mRNA delivery to cells compared to the standard formulation.

### Example 2. Optimizing Nucleic Acid Formulation with Chitosan/Cationic Lipids

Seven test formulations were prepared with the addition of different amounts of chitosan and DOTAP to the standard formulation (see Formulation Method A). For two of the test formulations, a modified formulation method (Formulation Method B) was used. The methods and materials used for preparing each of these formulations are detailed below.

Formulation Method A (Standard)

212.5 μL of 0.2 M sodium acetate, pH 7 was added to a sterile baked 2-mL vial. Next, 42.5 μL eGFP mRNA was added, and the solution was gently swirled by hand to mix. 425 μL of 132 mg/mL DOPC/Chol lipid vesicle particles (Size 81.02 nm, PDI 0.053, pH 6.77) prepared in DEPC treated water was added to this eGFP mRNA solution. The solution was gently swirled/shook by hand to mix and filled to 0.85 mL by adding 170 μL of DEPC treated water. The vial was then partially stoppered and freeze-dried. The freeze-dried cake was then reconstituted with 0.369 mL of Montanide® ISA 51 oil diluent to obtain final concentrations of DOPC/Chol 132 mg/mL, eGFP mRNA 0.1 mg/mL and sodium acetate 0.1 M. A 50 μL of this formulation was injected SC in mice.

Formulation Method A with Cationic Lipid DOTAP (66 mg/mL)

212.5 μL of 0.2 M sodium acetate, pH 7 was added to a sterile baked 2-mL vial. Next, 42.5 μL eGFP mRNA was added, and the solution was gently swirled by hand to mix. 212.5 μL of 132 mg/mL DOTAP lipid vesicle particles (Size 116.5 nm, PDI 0.125) prepared in DEPC treated water was added to this eGFP mRNA solution. The solution was gently swirled/shook by hand to mix and filled to 0.85 mL by adding 382.5 μL of DEPC treated water. The final pH of the formulation was pH 6.37. The vial was then partially stoppered and freeze-dried. The freeze-dried cake was then reconstituted with 0.397 mL of Montanide® ISA 51 oil diluent to obtain final concentrations of DOTAP 66 mg/mL, eGFP mRNA 0.1 mg/mL and sodium acetate 0.1 M. A 50 μL of this formulation was injected SC in mice.

Formulation Method A with Cationic Lipid DOTAP (132 mg/mL)

212.5 μL of 0.2 M sodium acetate, pH 7 was added to a sterile baked 2-mL vial. Next, 42.5 μL eGFP mRNA was added and the solution was gently swirled by hand to mix. 425 μL of 132 mg/mL DOTAP lipid vesicle particles (Size 116.5 nm, PDI 0.125) prepared in DEPC treated water was added to this eGFP mRNA solution. The solution was gently swirled/shook by hand to mix and filled to 0.85 mL by adding 170 μL of DEPC treated water. The final pH of the formulation was pH 6.37. The vial was then partially stoppered and freeze-dried. The freeze-dried cake was then reconstituted with 0.369 mL of Montanide® ISA 51 oil diluent to obtain final concentrations of DOTAP 66 mg/mL, eGFP mRNA 0.1 mg/mL and sodium acetate 0.1 M. A 50 μL of this formulation was injected SC in mice.

Formulation Method A with Chitosan Polymer (60-120 kDa/15-25% DD, 1 mg/mL)

212.5 μL of 0.2 M sodium acetate, pH 7 was added to a sterile baked 2-mL vial. Next, 42.5 μL eGFP mRNA was added and the solution was gently swirled by hand to mix. 212.5 μL of 132 mg/mL DOPC/Chol lipid vesicle particles (Size 81.02 nm, PDI 0.053, pH 6.77) prepared in DEPC treated water was added to this eGFP mRNA solution. The solution was gently swirled/shook by hand to mix. 42.5 μL of chitosan stock (60-120 kDa/15-25% DD, 10 mg/mL in 0.25% Acetic Acid) was added and the pH was adjusted from 5.91 to 6.15 with 10 μL 0.1 M NaOH. The solution was filled to 0.85 mL by adding 330 μL of DEPC treated water and freeze-dried. The freeze-dried cake was then reconstituted with 0.397 mL of Montanide® ISA 51 oil diluent to obtain final concentrations of DOPC/Chol 66 mg/mL, eGFP mRNA 0.1 mg/mL, chitosan 1 mg/mL and sodium acetate 0.1 M. A 50 μL of this formulation was injected SC in mice.

Formulation Method A with Chitosan Polymer (60-120 kDa/15-25% DD, 2 mg/mL)

212.5 μL of 0.2 M sodium acetate, pH 7 was added to a sterile baked 2-mL vial. Next, 42.5 μL eGFP mRNA was added and the solution was gently swirled by hand to mix. 212.5 μL of 132 mg/mL DOPC/Chol lipid vesicle particles (Size 81.02 nm, PDI 0.053, pH 6.77) prepared in DEPC treated water was added to this eGFP mRNA solution. The solution was gently swirled/shook by hand to mix. 85 μL of chitosan stock (60-120 kDa/15-25% DD, 10 mg/mL in 0.25% Acetic Acid) and adjusted the pH from 5.36 to 6.21 with 40 μL 0.1 M NaOH was added. The solution was filled to 0.85 mL by adding 257.5 μL of DEPC treated water and freeze-dried. The freeze-dried cake was then reconstituted with 0.397 mL of Montanide® ISA 51 oil diluent to obtain final concentrations of DOPC/Chol 66 mg/mL, eGFP mRNA 0.1 mg/mL, chitosan 2 mg/mL and sodium acetate 0.1 M. A 50 μL of this formulation was injected SC in mice.

Formulation Method A with Chitosan Polymer (100 kDa/25% DD, 1 mg/mL)

212.5 μL of 0.2 M sodium acetate, pH 7 was added to a sterile baked 2-mL vial. Next, 42.5 μL eGFP mRNA was added and the solution was gently swirled by hand to mix. 212.5 μL of 132 mg/mL DOPC/Chol lipid vesicle particles (Size 81.02 nm, PDI 0.053, pH 6.77) prepared in DEPC treated water was added to this eGFP mRNA solution. The solution was gently swirled/shook by hand to mix. 85 μL of chitosan stock (100 kDa/25% DD, 5 mg/mL in 0.25% Acetic Acid) was added and the pH was adjusted from 5.61 to 6.08 with 15 μL 0.1 M NaOH. The solution was filled to 0.85 mL by adding 282.5 μL of DEPC treated water and freeze-dried. The freeze-dried cake was then reconstituted with 0.397 mL of Montanide® ISA 51 oil diluent to obtain final concentrations of DOPC/Chol 66 mg/mL, eGFP mRNA 0.1 mg/mL, chitosan 1 mg/mL and sodium acetate 0.1 M. A 50 μL of this formulation was injected SC in mice.

Formulation Method B with Chitosan Polymer (100 kDa/25% DD, 1 mg/mL)

425 μL of 66 mg/mL DOPC/Chol solution prepared in DEPC treated water with 30% tert-butanol was added to a sterile baked 2-mL vial. Next, 42.5 μL eGFP mRNA was added. The solution was gently swirled by hand to mix. 85 μL of chitosan stock (100 kDa/25% DD, 5 mg/mL in 0.25% acetic acid) was added to this lipid-eGFP mRNA solution. The solution was gently swirled/shook by hand to mix and filled to 0.85 mL with 297.5 μL of 30% tert-butanol and freeze-dried. The freeze-dried cake was then reconstituted with 0.397 mL of Montanide® ISA 51 oil diluent to obtain final concentrations of DOPC/Chol 66 mg/mL, eGFP mRNA 0.1 mg/mL and chitosan 1 mg/mL. A 50 μL of this formulation was injected SC in mice.

Formulation Method B with Chitosan Polymer (100 kDa/25% DD, 2 mg/mL)

425 μL of 66 mg/mL DOPC/Chol solution prepared in DEPC treated water with 30% tert-butanol was added to a sterile baked 2-mL vial. Next, 42.5 μL eGFP mRNA was added. The solution was gently swirled by hand to mix. 170 μL of chitosan stock (100 kDa/25% DD, 5 mg/mL in 0.25% Acetic Acid) was added to this lipid-eGFP mRNA solution. The solution was gently swirled/shook by hand to mix and filled to 0.85 mL with 212.5 μL of 30% tert-butanol and freeze-dried. The freeze-dried cake was then reconstituted with 0.397 mL of Montanide® ISA 51 oil diluent to obtain final concentrations of DOPC/Chol 66 mg/mL, eGFP mRNA 0.1 mg/mL and chitosan 2 mg/mL. A 50 μL of this formulation was injected SC in mice.

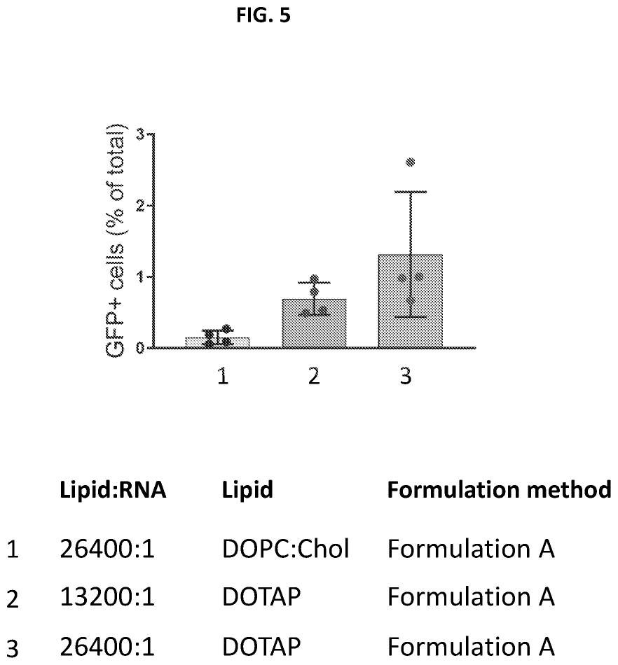

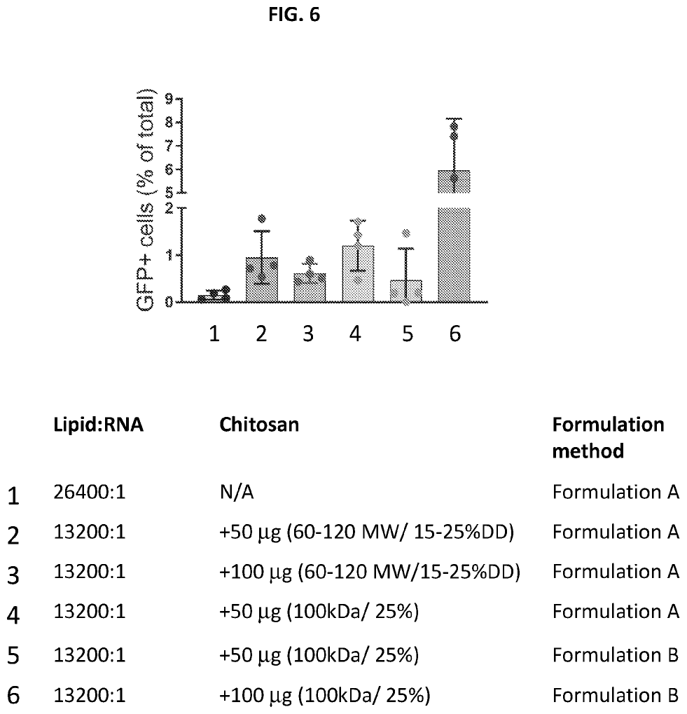

The expression of GFP in SOI cells was evaluated as described in Example 1. The results are shown in FIGS. 5 and 6. Consistent with previous observations, using DOTAP and addition of chitosan improved mRNA delivery to cells compared to the standard formulation, with the formulation having 100 μg chitosan polymer (100 kDa/25% DD) yielding the highest percentage of GFP+ cells.

### Example 3. Immunogenicity of Lipid-RNA Formulation

To evaluate immunogenicity of the lipid-RNA formulation, C57BL/6 mice were separated into 6 treatment groups (N=5/group) and received the respective lipid formulations listed in Table 1. On Day 0 of the study, Group 3 received the indicated lipid formulation via subcutaneous injection in the right flank. On Day 7, Groups 1, 2 and 4-6 received the respective lipid formulations via subcutaneous injection in the right flank. Mice were sacrificed on Day 15 followed by collection of cells from the SOI, spleen and right inguinal lymph nodes. The spleen and the lymph node samples were assessed for interferon-gamma (IFN-γ) release using an enzyme-linked immune absorbent spot (ELISpot) assay. The SOI samples were analyzed for the immune cell composition present in the sample using flow cytometry.

The ELISpot assay was performed as previously described (Weir et al, Oncoimmunology. 2014 Nov. 14; 3(8):e953407). Briefly, syngeneic dendritic cells (DCs) were prepared by culturing bone marrow derived cells with GM-CSF for 8 days. on study day 14 DCs were loaded with OVA peptide SIINFEKL (SEQ ID NO: 6) or an irrelevant peptide, or remained unloaded and used as antigen presenting cells for the ELISpot assay. On study day 15, single cell suspensions of lymph nodes were prepared in complete RPMI media. Lymph node cells and DCs were added to ELISpot plates (BD Bioscience) and incubated overnight at 37° C., 5% CO2. Plates were developed the next day as per the manufacturer's instructions using AEC substrate (Sigma-Aldrich). Spots were enumerated using an ImmunoSpot Analyzer (C.T.L. Ltd, Shaker Heights, OH) as the number of spot-forming units (SFU) per well.

An IFN-γ ELISpot performed using splenocytes had the following modifications. Single cell suspensions of splenocytes were prepared by lysing RBCs with ammonium-chloride-potassium solution; cells were added into ELISpot plates and stimulated with OVA peptide SIINFEKL (SEQ ID NO: 6) or an irrelevant peptide, or remained unstimulated.

Tissues from the site of injection were collected and processed by crushing in PBS buffer, centrifuging and resuspending in PBS buffer. Cells were stained using basic surface IMF staining protocol and antibody panel in Table 4. Multi-parametric flow cytometry analysis was performed using FACS Celesta and FlowJo software.

TABLE 1
Treatment Groups

| | | Vaccinate/ | |
|---|---|---|---|
| Group | N | ELISpot (day) | Formulation |
| 1 | 5 | 7/15 | DOPC:Chol - OVA peptide |
| 2 | 5 | 7/15 | DOTAP - OVA mRNA |
| 3 | 5 | 0/15 | DC:Chol - OVA mRNA |
| 4 | 5 | 7/15 | DOPC:Chol with 60-120 kDa/15- |
| | | | 25% DD Chitosan (Formulation |
| | | | Method A) OVA mRNA |
| 5 | 5 | 7/15 | DOPC:Chol with 100 kDa/25% |
| | | | DD Chitosan (Formulation |
| | | | Method A) - OVA mRNA |
| 6 | 5 | 7/15 | DOPC:Chol with 100 kDa/25% |
| | | | DD Chitosan (Formulation |
| | | | Method B)- OVA mRNA |

The composition of the formulations listed in Table 1 is detailed in Tables 2A and 2B. mRNA containing formulations were prepared using OVA mRNA stock according to specifications described in the previous example.

TABLE 2A
Lipid-mRNA Formulations

| | DOPC:Chol- | | DOPC:Chol- | | | | | | DOPC:Chol- | |
|---|---|---|---|---|---|---|---|---|---|---|
| | 66 mg-100 | | 66 mg- 60-120 | | | | | | 66 mg 100 | |
| | kDa/25% | | kDa/15-25% | | | | | | kDa/25% | |
| | DD chitosan | | DD chitosan | | DOTAP- | | DC-Chol- | | DD chitosan | |
| Formulations | (100 μg) | | (50 μg) | | 66 mg | | 66 mg | | (50 μg) | |
| Dose Volume | 50 | μL | 50 | μL | 50 | μL | 50 | μL | 50 | μL |
| OVA mRNA/ | 5 | μg | 5 | μg | 5 | μg | 5 | μg | 5 | μg |
| dose | | | | | | | | | | |

| | | | | | |
|---|---|---|---|---|---|
| Adjuvant/ | 100 μg | 50 μg | — | — | 50 μg |
| delivery | 100 kDa/25% | 60-120 kDa/ | | | 100 kDa/25% |
| helper | DD chitosan | 15-25% DD | | | DD chitosan |
| (per dose) | | chitosan | | | |
| Lipid | DOPC:Chol | DOPC:Chol | DOTAP | DC-Chol | DOPC:Chol |
| concentration | (66 mg/mL) | (66 mg/mL) | (66 mg/mL) | (66 mg/mL) | (66 mg/mL) |
| Oil/diluent | Montanide ® | Montanide ® | Montanide ® | Montanide ® | Montanide ® |
| | ISA 51VG | ISA 51VG | ISA 51VG | ISA 51VG | ISA 51VG |
| Method | Formulation | Formulation | Formulation | Formulation | Formulation |
| | B | A | A | A | A |

TABLE 2B
Lipid-peptide Control

| Formulations | Lipid-OVA peptide | |
|---|---|---|
| Dose Volume | 50 | μL |
| OVA/dose | 50 | μg |
| Adjuvant/delivery helper (per dose) | 20 | μg dIdC |

| | |
|---|---|
| Lipid concentration | DOPC:Chol (132 mg/mL) |
| Oil/diluent | Montanide ISA 51VG |
| Method | Formulation A |

Lipid-peptide control formulation listed in Table 2B was prepared by adding 80 μL of OVA peptide stock (5 mg/me in sterile RNase free water) to 200 μL sodium acetate 200 mM, pH 7.0. To the diluted peptide stock solution, 400 μL lipid vesicle particles (132 mg/mL DOPC/Chol prepared in sterile RNase free water, particle size 89.27 nm and PDI 0.088) was added, mixed well by vortexing for 30 seconds. The pH was adjusted to 7.03 with 0.1 M NaOH before the addition of 16 μL poly dIdC adjuvant stock (10 mg/mL in sterile RNase free water). The final formulation volume was then filled to 0.8 mL by adding 97 μL of RNase free water, mixed well by vortexing for 30 seconds. The vial was then partially stoppered and freeze-dried. The freeze-dried cake was then reconstituted with 0.35 mL of Montanide ISA 51 oil diluent to obtain final concentrations of DOPC/Chol 132 mg/m, OVA peptide 1 mg/mL, dIdC adjuvant 0.4 mg/mL and sodium acetate 0.1 M. A 50 μL of this formulation was injected subcutaneously (SC) in mice.

Table 3 below lists the material used in the experiments above and their sources.

TABLE 3
Materials

| Material Type | Description | Source |
|---|---|---|
| Antigen | OVA mRNA* | CleanCap ®Ovalbumin mRNA |
| | | (TriLink BioTechnologies |
| | | #L-7610-100) |
| Antigen | OVA peptide | GenScript |
| | SIINFEKL | |
| | (SEQ ID NO: 6) | |
| Adjuvant | dIdC | Biospring |
| | Chitosan | Mycodev (100 kDa/25% DD), |
| | | Invivogen (60-120 kDa/ |
| | | 15-25% DD) |
| Lipids | DOPC/Chol mixture | Lipoid |
| | DOTAP | |
| | DC:Chol | |
| Oil/Diluent | Montanide ISA 51 VG | SEPPIC |

*OVA mRNA has 1437 nucleotides and encodes the full-length ovalbumin.

TABLE 4
IMF panel to profile immune cells at the site of injection

| | Markers/ | | |
|---|---|---|---|
| Characterized Cells | Antibodies | Color | Clone |
| Immune Cells (CD45+) | CD45 | APC Cy7 | 30-F11 |
| T Cells (CD3+) | CD3 | BV605 | 17A2 |
| Cytotoxic T cells (CD8+) | CD8 | PerCP | 53-6.7 |
| Helper T cells (CD4+) | CD4 | BV510 | GK1.5 |
| NK Cells (NK1.1) | NK1.1 | PE-CF594 | PK136 |
| B Cells (CD19+) | CD19 | BV421 | 1D3 |
| Dendritic Cells (CD11c+, MHC | CD11c | AF700 | HL3 |
| II+) | MHC II | FITC | M5/114.15.2 |
| Macrophages (CD11b+, F4/80+) | CD11b | BV786 | M1/70 |
| | F4/80 | PE | BM8 |
| Neutrophils (CD11b+, Ly6G+) | Ly6G | APC | 1A8Ly6G |

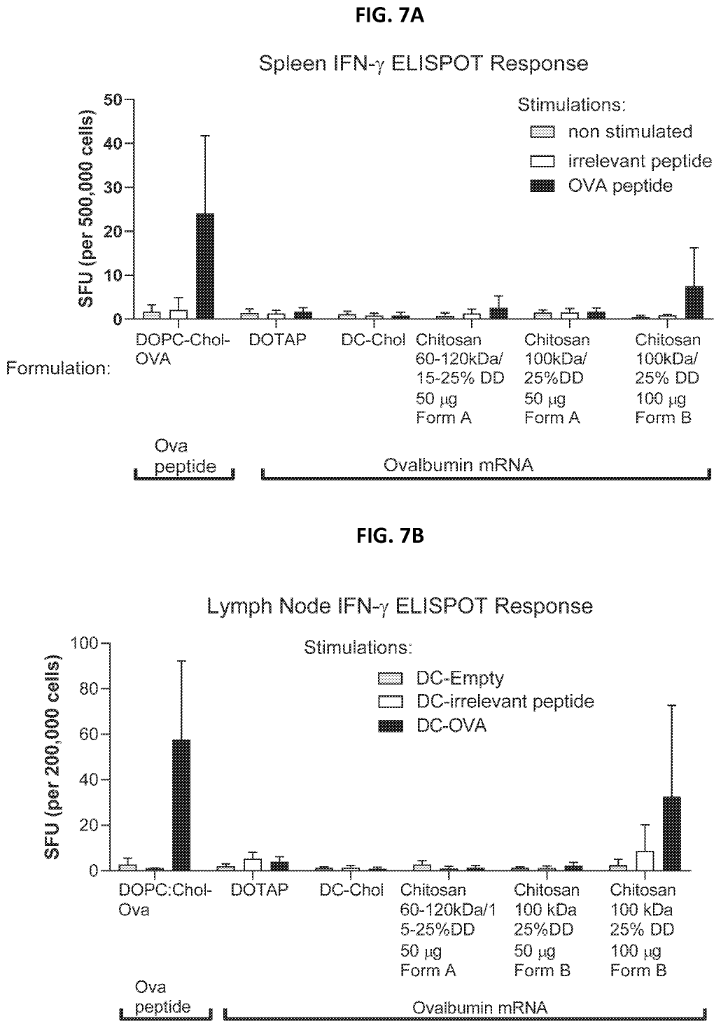

The spleen and lymph node IFN-γ ELISpot response are shown in FIGS. 7A and 7B, respectively. Single immunization with lipid formulations containing Ovalbumin mRNA and modified with chitosan polymer induced detectable immune responses in draining lymph nodes and spleens 8 days post administration. Consistently, results of the previous experiment (see FIG. 6) indicate that this formulation delivers the highest number of mRNA molecules to the cells at the SOI.

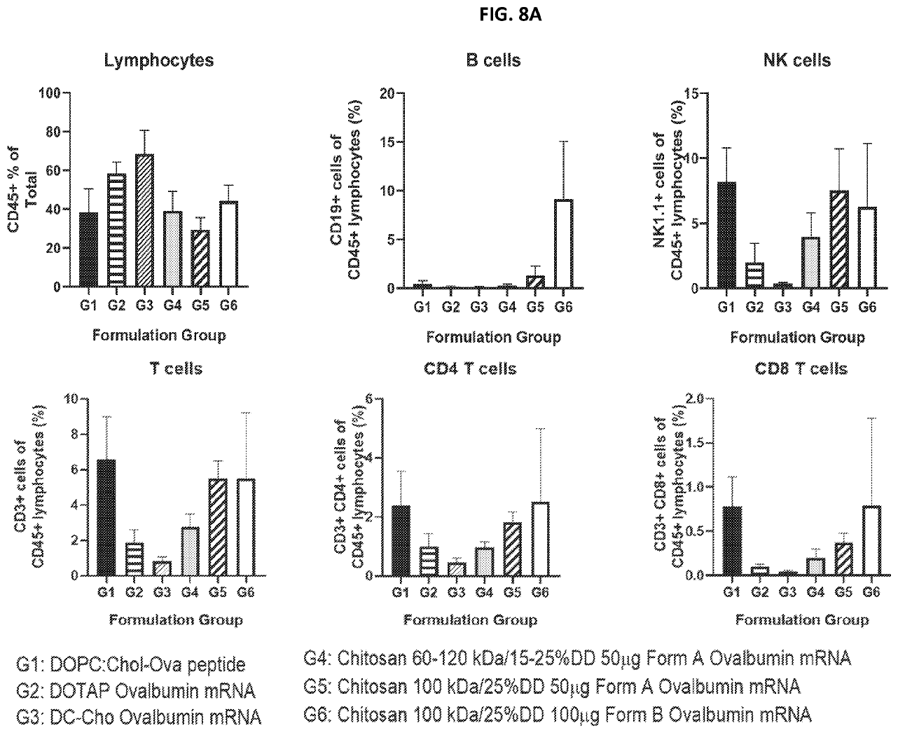

Immune profile of SOIs is shown in FIGS. 8A and 8B. In general, lipid formulation modified with chitosan attracted similar types of immune cells as did the standard lipid-peptide formulation, suggesting a potentially similar mechanism of action (MOA). However, differently from lipid-peptide formulation, lipid formulation modified with chitosan surprisingly attracted higher number of B cells. Lipid formulation modified with DC-Chol and DOTAP attracted different types of immune cells compared to the standard lipid-peptide formulation, suggesting differences in MOA.

### Example 4. In Vitro and In Vivo Stability of Lipid-Nucleic Acid Formulations

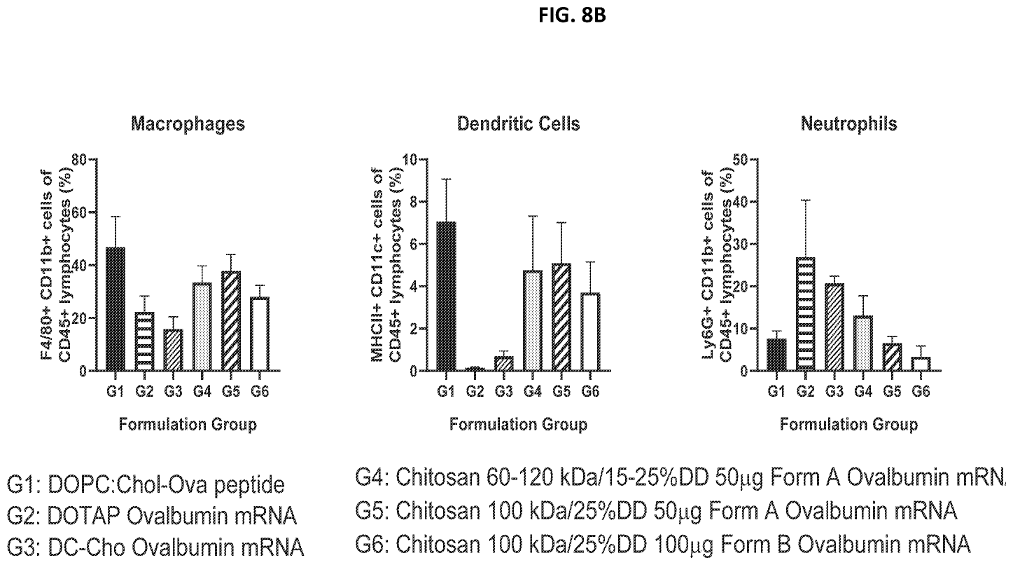

In vitro stability testing of mRNA and DNA formulated in the lipid formulation was carried our as outlined in FIG. 9. The lipid-RNA containing eGFP mRNA or lipid-DNA formulation containing pGFP plasmid DNA or lipid formulation without mRNA or DNA (negative control Empty) was prepared using Formulation Method A described in Examples 1 or 2 and reconstituted in Montanide® ISA 51 oil diluent. The oil reconstituted sample was then stored at 37° C. for two weeks. On each study day D=0, 1, 3, 7, 10, 14, 25 μL aliquot of the stored oil reconstituted sample was removed from each group for the nucleic acid extraction and analysis. Briefly, nucleic acid extraction was carried out as follows: aliquoted sample (25 μL) was diluted with 0.1 M sodium bicarbonate (50 μL). To this diluted sample, water-saturated 1-butanol (75 μL) was added, vortexed for 15 seconds and centrifuged for 2 minutes at 5,500 rpm. Using a gel-loading pipette tip, a portion of the bottom layer was collected for nucleic acid analysis. Nucleic acid quantity was measured by UV spectroscopy. mRNA and DNA integrity was assessed by gel electrophoresis assay. mRNA and DNA expression efficiency was evaluated in transient transfection experiments.

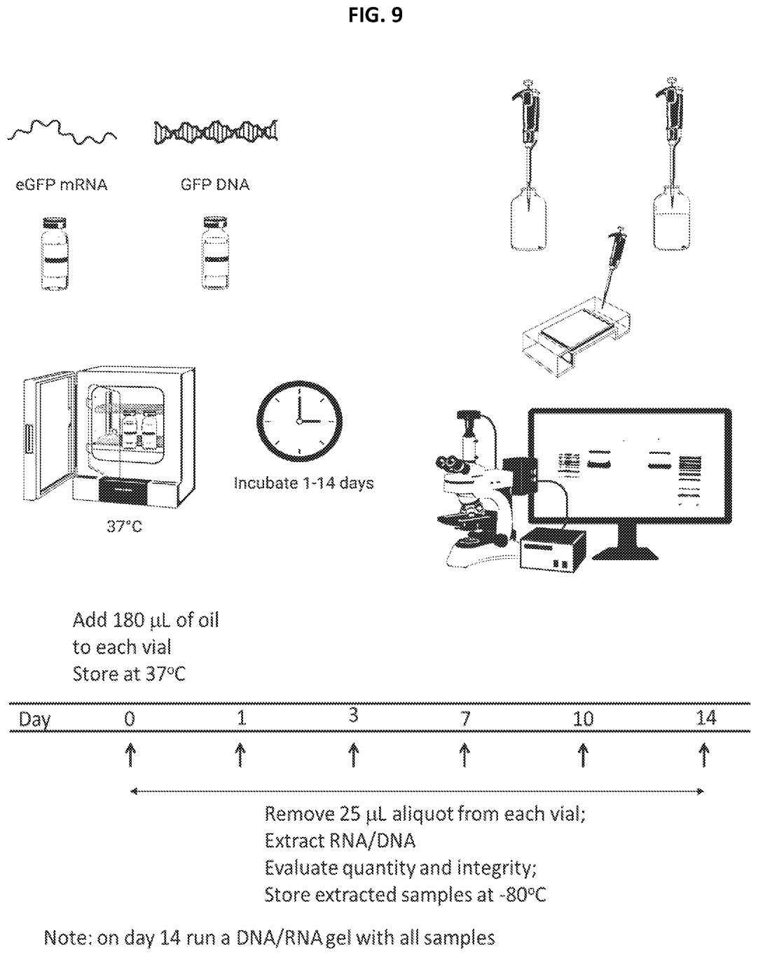

Nucleic acid quantity as measured by UV spectroscopy using NanoDrop OneC spectrophotometer is shown in FIG. 10A for mRNA and in FIG. 10B for DNA. The data accompanying the figures are also shown in Tables 5A and 5B. No substantial changes in nucleic acid quantity was observed over time.

TABLE 5A
mRNA quantity

| Sample | Expected Concentration | | Actual Concentration | |
|---|---|---|---|---|
| eGFP mRNA D0 | 0.5 | μg/μL | 0.41069 | μg/μL |
| eGFP mRNA D1 | 0.5 | μg/μL | 0.46395 | μg/μL |
| eGFP mRNA D3 | 0.5 | μg/μL | 0.70802 | μg/μL |
| eGFP mRNA D7 | 0.5 | μg/μL | 0.331885 | μg/μL |
| eGFP mRNA D10 | 0.5 | μg/μL | 0.315605 | μg/μL |
| eGFP mRNA D14* | 0.5 | μg/μL | 0.911575 | μg/μL |

TABLE 5B
DNA quantity

| Sample | Expected Concentration | | Actual Concentration | |
|---|---|---|---|---|
| pGFP DNA D0 | 0.5 | μg/μL | 0.44406 | μg/μL |
| pGFP DNA D1 | 0.5 | μg/μL | 0.37862 | μg/μL |
| pGFP DNA D3 | 0.5 | μg/μL | 0.38573 | μg/μL |
| pGFP DNA D7 | 0.5 | μg/μL | 0.33434 | μg/μL |
| pGFP DNA D10 | 0.5 | μg/μL | 0.496405 | μg/μL |
| pGFP DNA D14* | 0.5 | μg/μL | 1.03105 | μg/μL |

*Day 14 had a deviation in extraction procedure that affected the quantity of nucleic acids extracted.

To evaluate DNA integrity, 0.5 μg of pGFP DNA after butanol extraction was resolved on a 1% TBE agarose gel, visualized by staining with EtBr and photographed. Intact pGFP DNA was loaded on the same gel and used as a positive control.

To evaluate RNA integrity, 1 μg of eGFP mRNA after butanol extraction was resolved on 0.8% agarose denaturing gel, visualized by staining with ethidium bromide and photographed. Intact eGFP mRNA was loaded on the same gel and used as a positive control.

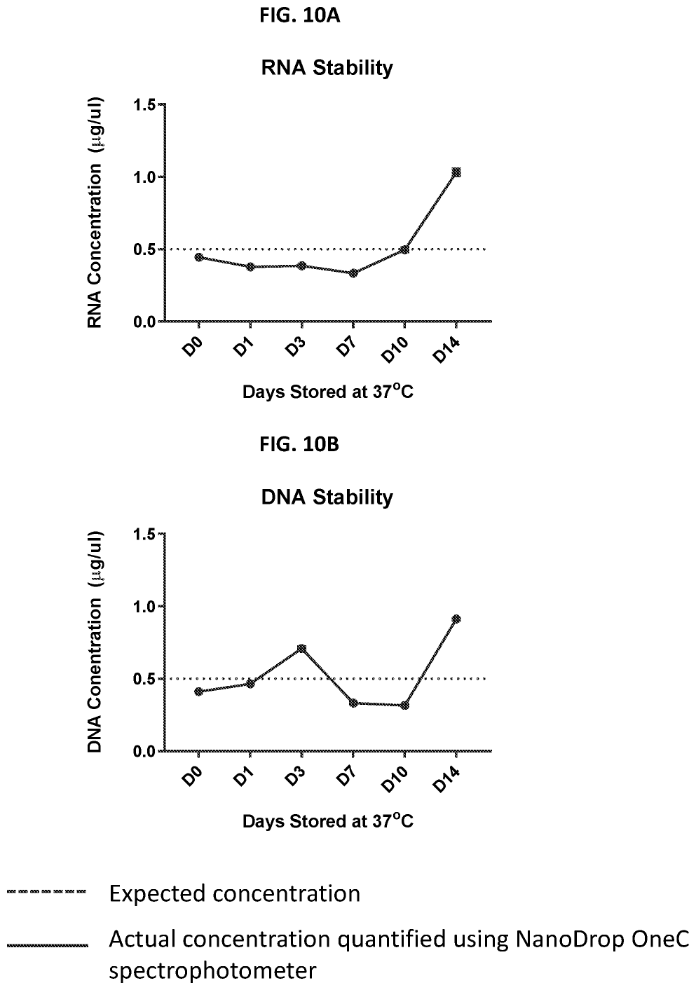

Gel electrophoresis analyses of mRNA and DNA integrity are shown in FIGS. 11A and 11B, respectively. No significant changes were observed in the mRNA integrity in any of the extracted samples as compared to the intact eGFP mRNA control. Some smears below the intact eGFP mRNA band were visible at day 10 and 14, suggesting some degree of degradation in these samples. Apparent changes in the pGFP DNA conformation were detected from DO to D14; these may include gradual reduction of the supercoiled form (intact plasmid conformation) and gradual increase of the linear and nicked forms (degraded conformations).

To further confirm nucleic acid integrity a functional test of gene expression was conducted through a transfection assay on the collected samples. To confirm that extracted eGFP mRNA is capable of producing functional protein 293T cells were transfected with 2.5 μg of extracted eGFP mRNA using Lipofectamine™ Messenger MAX™ Transfection Reagent (Invitrogen).

To confirm that extracted pGFP DNA is capable of producing functional protein 293T cells were transfected with 0.5 μg of extracted pGFP DNA using Lipofectamine™ 2000 Transfection Reagent (Invitrogen).

48 hours after transfection cells were harvested and flow cytometry analysis was performed using FACS Celesta and FlowJo software to evaluate GFP expression.

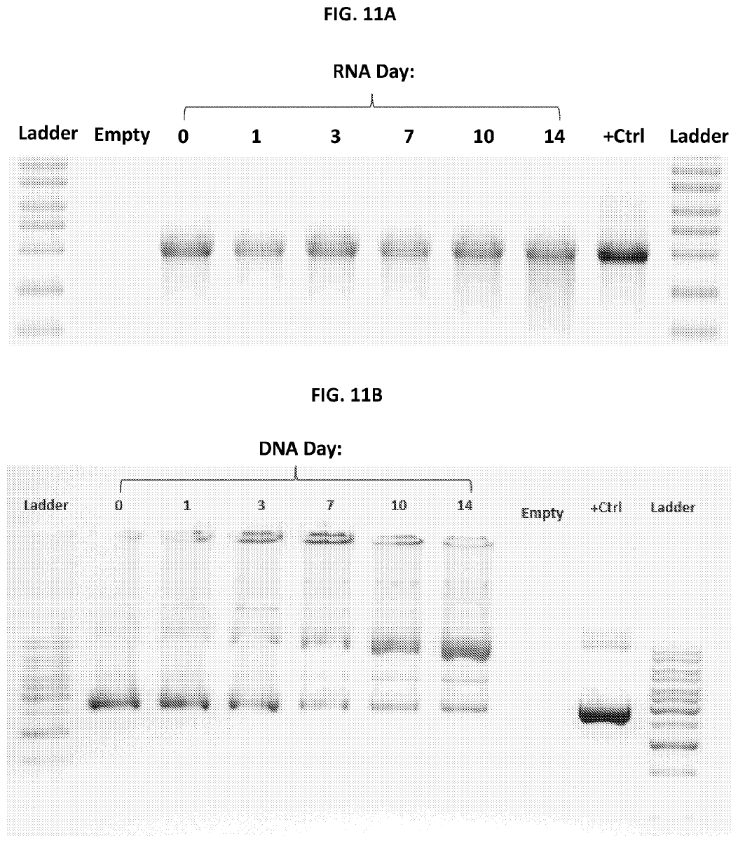

Results from the transient transfection experiments are presented in FIG. 12. The percentage of GFP⁺ positive cells remained consistent from D0 to D14 (99%±1% for eGFP mRNA and 44%±9% for pGFP plasmid) as measured using flow cytometry. The mean fluorescence intensities show a decreasing trend from D0 to D14, suggesting that the total number of pGFP plasmids and eGFP mRNA molecules per cell was decreased over time.

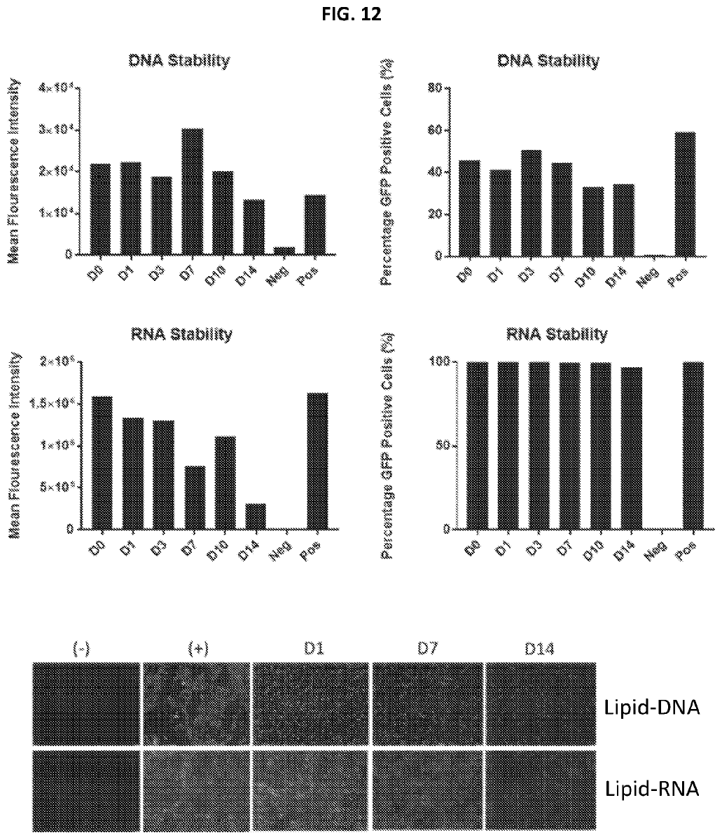

In vivo stability testing of mRNA formulated in lipid-RNA formulation was carried out as outlined in FIG. 13. On Day 0, mice were immunized SC with a mix of E7 and eGFP mRNA formulated in lipid formulation or with lipid formulation containing no RNA (negative control, Empty). Formulations were prepared using Formulation Method A described in Examples 1 or 2 and reconstituted in Montanide® ISA 51 oil diluent. On Days 7, 14 and 21, SOIs were collected and processed for mRNA extraction.

Total mRNA was purified using RNeasy Mini Kit (Qiagen) and used in RT-PCR to evaluate mRNA integrity and in transient transfections to determine the capacity of the extracted RNA to produce functional protein.

The complete sequence of the E7 transcript was amplified via RT-PCR. Briefly, extracted RNA was quantified using a Nanodrop and processed for reverse transcription of eluted RNA to cDNA. Template cDNA was then used in PCR to amplify E7 and GAPDH transcripts (positive control; data not shown) using primers listed in Table 6. E7 transcript was amplified using primers specific to the 5′ and 3′ ends of E7 mRNA. Amplifications were performed using the HotStarTaq DNA Polymerase kit (Qiagen) according to the manufacturer's instructions.

TABLE 6
Primers for amplification of E7
and GAPDH transcripts

| | | Forward | Reverse | Product |
|---|---|---|---|---|
| | Amplicon | Primer | Primer | size |
| 1 | E7 | E7-S4 | E7-AS4 | 309 bp |
| | complete | 5′GAATTC | 5′CATGTCG | |
| | sequence | ATGCATGG | AGCTAGCTT | |
| | | AGATACAC | ATGGTTTCT | |
| | | CTACATTG | GAGAACAG | |
| | | CA-3′ | ATGG-3′ | |
| | | (SEQ ID | (SEQ ID | |
| | | NO: 7) | NO: 8) | |
| 2 | GAPDH | GAPDH-S2 | GAPDH-AS2 | 218 bp |
| | | 5′GACGGC | 5′TGTGCCG | |
| | | CGCATCTT | TTGAATTTG | |
| | | CTTGTG-3′ | CCGT-3′ | |
| | | (SEQ ID | (SEQ ID | |
| | | NO: 9) | NO: 10) | |

PCRs were performed in a thermocycler using the following conditions: initial denaturing (95° C.) for 15 min, 40 cycles of denaturing (94° C.) for 1 min, annealing (57° C.) for 30 sec, and elongation (72° C.) for 1 min. PCR products were resolved on a 1% agarose gel, visualized by staining with ethidium bromide and photographed.

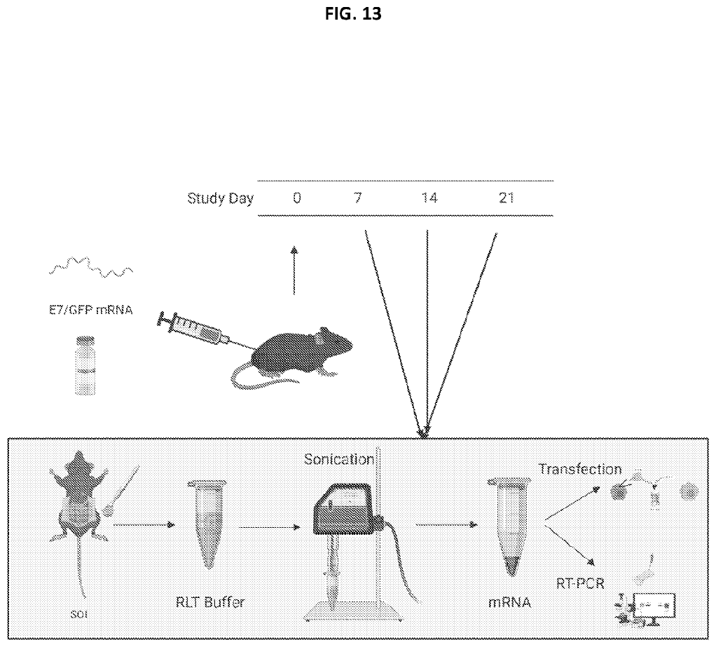

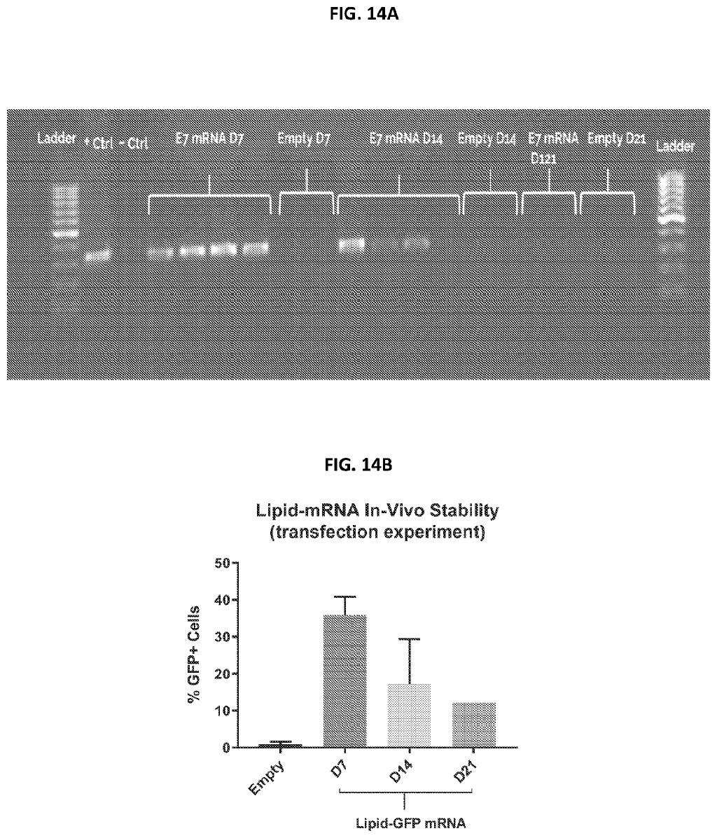

The results are shown in FIG. 14A. Complete sequence of the E7 transcript was amplified from total RNA extracted from SOIs at SD7 and 14.

To determine the capacity of eGFP mRNA after formulation and incubation in mice to produce functional protein 5 μg of total RNA extracted from SOIs was used to transfect 293T cells using Lipofectamine™ Messenger MAX™ Transfection Reagent (Invitrogen). 48 hours after transfection cells were harvested and flow cytometry analysis was performed using FACS Celesta and FlowJo software to evaluate GFP expression.

The results are shown in FIG. 14B. The percentage of GFP+ cells was assessed in control group containing no mRNA and was 0.92%. The percentage of GFP+ cells in samples collected from SOIs of mice vaccinated with lipid-RNA formulation containing eGFP mRNA collected at D7 was 35.8%, at D14 was 17.3% and at D21 was 12.2%. This suggests that at least some eGFP mRNA molecules formulated in lipid formulation remain intact and capable to produce functional protein after 21 days inoculation in vivo.

Together results of integrity test by RT-PCR and functional analysis by transient transfection demonstrate that mRNA is stable in the lipid-RNA formulation for up to 14 days in vivo.

RNA instability is known to be one of the factors that hinder the application of RNA-based therapeutics. Exogenous RNA tends to be rapidly eliminated from the body due to degradation by RNase, exonuclease, or endonuclease, or via renal or hepatic clearance. The studies described in this Example showed that intact mRNA and plasmid DNA are present in the lipid formulations after 14-day incubation at 37° C. in vitro, however the integrity of mRNA and DNA gradually decreases. mRNA is stable in vivo in the lipid formulation for up to 14 days as assessed by RT-PCR and transfection. As a result of these two studies, it can be concluded that mRNA formulated in the lipid formulation described herein is stable for at least 14 days at 37° C. in vitro and in vivo.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A composition, comprising:
a) one or more lipids,
b) a negatively charged molecule,
c) a carrier comprising a continuous phase of a hydrophobic substance, and
d) an ionizable aminoglycoside.

2. The composition of claim 1, wherein the one or more lipids comprise one or more of a phospholipid, cholesterol or a cholesterol derivative, or a combination thereof.

3. (canceled)

4. The composition of claim 2, wherein the phospholipid comprises dioleoyl phosphatidylcholine (DOPC), 1,2-dipalmitoyl-sn-glycero-3-phosphocholine (DPPC), dioleoyl phosphatidylethanolamine (DOPE), 1,2-dipalmitoyl-sn-glycero-3-succinate (DGS), 1,2-dioleoyl-3-trimethylammonium-propane (DOTAP), 3β-[N—(N′,N′-dimethylamino ethane)-carbamoyl]cholesterol (DC-cholesterol), 1,2-distearoyl-3-dimethylammonium-propane (DAP), N-(4-carboxybenzyl)-N,N-dimethyl-2,3-bis(oleoyloxy)propan-1-aminium (DOBAQ), N-palmitoyl homocysteine (PHC), or a combination thereof.

5. The composition of claim 1, wherein the one or more lipids comprise DOPC and cholesterol.

6. The composition of claim 1, wherein the ionizable aminoglycoside is one or more of chitosan, cationic alginate, cationic gelatin, cationic dextran, diethylaminoethyl (DEAE)-dextran hydrochloride, aminated cellulose, aminated sucrose, aminated trehalose, N-acetyl-D-glucosamine, D-(+)-glucosamine hydrochloride, trehalose-6,6-dibehenate (TDB) with Dimethyldioctadecylammonium (DDA), heptakis(6-deoxy-6-amino)-β-cyclodextrin heptahydrochloride, and glycyrrhizic acid ammonium salt, or derivatives thereof.

7. The composition of claim 1, wherein the ionizable aminoglycoside is chitosan.

8. The composition of claim 7, wherein:
the chitosan has a molecular weight of about 60 kDa to 150 kDa, about 100 kDa to 120 kDa, or about 100 Da; and/or
the chitosan has a degree of deacetylation (DD) of about 15-95%, or about 25%.

9-10. (canceled)

11. The composition of claim 1, wherein:
the negatively charged molecule is a polynucleotide comprising DNA; or
the negatively charged molecule is a polynucleotide comprising RNA, wherein the RNA is a messenger RNA (mRNA), an antisense RNA, an interfering RNA, a catalytic RNA, or a ribozyme.

12-15. (canceled)

16. The composition of claim 1, wherein the carrier comprises an oil or a water-in-oil emulsion.

17. The composition of claim 1, wherein
a) the one or more lipids comprise DOPC and cholesterol,
b) the negatively charged molecule is a polynucleotide,
c) the carrier comprises an oil or a water-in-oil emulsion, and
d) the ionizable aminoglycoside is chitosan.

18. (canceled)

19. The composition of claim 17, wherein the carrier is a mannide oleate in mineral oil solution.

20-22. (canceled)

23. A method for delivering a negatively charged molecule to a subject, comprising administering the composition of claim 1 to said subject.

24. A method for treating or preventing cancer, an infectious disease or a disease and/or disorder ameliorated by humoral and/or cellular immune response in a subject in need thereof, said method comprising administering to the subject an effective amount of the composition of claim 1.

25. (canceled)

26. A method for preparing a composition comprising one or more lipids, a negatively charged molecule, a carrier comprising a continuous phase of a hydrophobic substance, and an ionizable aminoglycoside, comprising:
a) dissolving the one or more lipids in one or more organic solvents and optionally an aqueous solvent to create a lipid solution,
b) adding the negatively charged molecule to the lipid solution formed in step a) and mixing;
c) adding the ionizable aminoglycoside to the mixture formed in step b) and mixing;
d) optionally, adding additional amount of the organic solvent(s) or aqueous solvent to the mixture formed in step c) thereby the overall Wt/Wt or V/V percentage ratio of organic:aqueous solvent or aqueous:organic solvent in the mixture is between 20-50%;
e) drying the mixture formed in step c) or d) to generate a dried preparation; and
f) dissolving the dried preparation in the carrier comprising a continuous phase of a hydrophobic substance, thereby generating said composition.

27. The method of claim 26, wherein in step a) the one or more organic solvents is present in an amount sufficient to prevent the one or more lipids from forming lipid vesicle particles in the lipid solution.

28. The method of claim 26, wherein the one or more organic solvents comprises tert-butanol, ethanol, methanol, chloroform, or a mixture thereof.

29-30. (canceled)

31. The method of claim 26, wherein the one or more lipids comprise one or more of a phospholipid, cholesterol or a cholesterol derivative, or a combination thereof.

32. (canceled)

33. The method of claim 31, wherein the phospholipid comprises dioleoyl phosphatidylcholine (DOPC), 1,2-dipalmitoyl-sn-glycero-3-phosphocholine (DPPC), dioleoyl phosphatidylethanolamine (DOPE), 1,2-dipalmitoyl-sn-glycero-3-succinate (DGS), 1,2-dioleoyl-3-trimethylammonium-propane (DOTAP), 3β-[N—(N′,N′-dimethylamino ethane)-carbamoyl]cholesterol (DC-cholesterol), 1,2-distearoyl-3-dimethylammonium-propane (DAP), N-(4-carboxybenzyl)-N,N-dimethyl-2,3-bis(oleoyloxy)propan-1-aminium (DOBAQ), N-palmitoyl homocysteine (PHC), or a combination thereof.

34. The method of claim 26, wherein the one or more lipids comprise DOPC and cholesterol.

35. The method of claim 26, wherein the ionizable aminoglycoside is one or more of chitosan, cationic alginate, cationic gelatin, cationic dextran, diethylaminoethyl (DEAE)-dextran hydrochloride, aminated cellulose, aminated sucrose, aminated trehalose, N-acetyl-D-glucosamine, D-(+)-glucosamine hydrochloride, trehalose-6,6-dibehenate (TDB) with Dimethyldioctadecylammonium bromide (DDA), heptakis(6-deoxy-6-amino)-β-cyclodextrin heptahydrochloride, and glycyrrhizic acid ammonium salt, or derivatives thereof.

36. The method of claim 35, wherein the ionizable aminoglycoside is chitosan.

37. The method of claim 35, wherein;
the chitosan has a molecular weight of about 60 kDa to 150 kDa, about 100 kDa to 120 kDa, or 100 kDa; and/or
the chitosan has a degree of deacetylation (DD) of about 15-95%, or about 25%.

38-39. (canceled)

40. The method of claim 26, wherein:
the negatively charged molecule is a polynucleotide comprising DNA; or
the negatively charged molecule is a polynucleotide comprising RNA, wherein the RNA is a messenger RNA (mRNA), an antisense RNA, an interfering RNA, a catalytic RNA, or a ribozyme.

41-45. (canceled)

46. The method of claim 26, wherein
a) the one or more lipids comprise DOPC and cholesterol,
b) the negatively charged molecule is a polynucleotide,
c) the carrier comprises an oil or a water-in-oil emulsion, and
d) the ionizable aminoglycoside is chitosan.

47. (canceled)

48. The method of claim 46, wherein the carrier comprises a mannide oleate in mineral oil solution.

49-50. (canceled)

51. A composition produced by the method of claim 26.
